SEQUENCE LISTING (9 sequences)

SEQ ID NO: 1 - protein, 419 aa, Human
MLAAAFADSNSSSMNVSFAHLHFAGGYLPSDSQDWRTIIPALLVAVCLVGFVGNLCVIGILLHNAWKGKPSMIHSLILNLSLADLSLLLFSAPIRATAYSKSVWDLGWFVCKSSDWFIHTCMAAKSLTIVVVAKVCFMYASDPAKQVSIHNYTIWSVLVAIWTVASLLPLPEWFFSTIRHHEGVEMCLVDVPAVAEEFMSMFGKLYPLLAFGLPLFFASFYFWRAYDQCKKRGTKTQNLRNQIRSKQVTVMLLSIAIISALLWLPEWVAWLWVWHLKAAGPAPPQGFIALSQVLMFSISSANPLIFLVMSEEFREGLKGVWKWMITKKPPTVSESQETPAGNSEGLPDKVPSPESPASIPEKEKPSSPSSGKGKTEKAEIPILPDVEQFWHERDTVPSVQDNDPIPWEHEDQETGEGVK

SEQ ID NO: 2 - DNA, 1257 bp, Human
atgctggcagctgcctttgcagactctaactccagcagcatgaatgtgtcctttgctcacctccactttgccggagggtacctgccctctgattcccaggactggagaaccatcatcccggctctcttggtggctgtctgcctggtgggcttcgtgggaaacctgtgtgtgattggcatcctccttcacaatgcttggaaaggaaagccatccatgatccactccctgattctgaatctcagcctggctgatctctccctcctgctgttttctgcacctatccgagctacggcgtactccaaaagtgtttgggatctaggctggtttgtctgcaagtcctctgactggtttatccacacatgcatggcagccaagagcctgacaatcgttgtggtggccaaagtatgcttcatgtatgcaagtgacccagccaagcaagtgagtatccacaactacaccatctggtcagtgctggtggccatctggactgtggctagcctgttacccctgccggaatggttctttagcaccatcaggcatcatgaaggtgtggaaatgtgcctcgtggatgtaccagctgtggctgaagagtttatgtcgatgtttggtaagctctacccactcctggcatttggccttccattattttttgccagcttttatttctggagagcttatgaccaatgtaaaaaacgaggaactaagactcaaaatcttagaaaccagatacgctcaaagcaagtcacagtgatgctgctgagcattgccatcatctctgctctcttgtggctccccgaatgggtagcttggctgtgggtatggcatctgaaggctgcaggcccggccccaccacaaggtttcatagccctgtctcaagtcttgatgttttccatctcttcagcaaatcctctcatttttcttgtgatgtcggaagagttcagggaaggcttgaaaggtgtatggaaatggatgataaccaaaaaacctccaactgtctcagagtctcaggaaacaccagctggcaactcagagggtcttcctgacaaggttccatctccagaatccccagcatccataccagaaaaagagaaacccagctctccctcctctggcaaagggaaaactgagaaggcagagattcccatccttcctgacgtagagcagttttggcatgagagggacacagtcccttctgtacaggacaatgaccctatcccctgggaacatgaagatcaagagacaggggaaggtgttaaa

SEQ ID NO: 3 - protein, 419 aa, Human
MLAAAFADSNSSSMNVSFAHLHFAGGYLPSDSQDWRTIIPALLVAVCLVGFVGNLCVIGILLHNAWKGKPSMIHSLILNLSLADLSLLLFSAPIRATAYSKSVWDLGWFVCKSSDWFIHTCMAAKSLTIVVVAKVCFMYASDPAKQVSIHNYTIWSVLVAIWTVASLLPLPEWFFSTIRHHEGVEMCLVDVPAVAEEFMSMFGKLYPLLAFGLPLFFASFYFWRAYDQCKKRGTKTQNLRNQIRSKQVTVMLLSIAIISAVLWLPEWVAWLWVWHLKAAGPAPPQGFIALSQVLMFSISSANPLIFLVMSEEFREGLKGVWKWMITKKPPTVSESQETPAGNSEGLPDKVPSPESPASIPEKEKPSSPSSGKGKTEKAEIPILPDVEQFWHERDTVPSVQDNDPIPWEHEDQETGEGVK

SEQ ID NO: 4 - DNA, 1257 bp, Human
atgctggcagctgcctttgcagactctaactccagcagcatgaatgtgtcctttgctcacctccactttgccggagggtacctgccctctgattcccaggactggagaaccatcatcccggctctcttggtggctgtctgcctggtgggcttcgtgggaaacctgtgtgtgattggcatcctccttcacaatgcttggaaaggaaagccatccatgatccactccctgattctgaatctcagcctggctgatctctccctcctgctgttttctgcacctatccgagctacggcgtactccaaaagtgtttgggatctaggctggtttgtctgcaagtcctctgactggtttatccacacatgcatggcagccaagagcctgacaatcgttgtggtggccaaagtatgcttcatgtatgcaagtgacccagccaagcaagtgagtatccacaactacaccatctggtcagtgctggtggccatctggactgtggctagcctgttacccctgccggaatggttctttagcaccatcaggcatcatgaaggtgtggaaatgtgcctcgtggatgtaccagctgtggctgaagagtttatgtcgatgtttggtaagctctacccactcctggcatttggccttccattattttttgccagcttttatttctggagagcttatgaccaatgtaaaaaacgaggaactaagactcaaaatcttagaaaccagatacgctcaaagcaagtcacagtgatgctgctgagcattgccatcatctctgctgtcttgtggctccccgaatgggtagcttggctgtgggtatggcatctgaaggctgcaggcccggccccaccacaaggtttcatagccctgtctcaagtcttgatgttttccatctcttcagcaaatcctctcatttttcttgtgatgtcggaagagttcagggaaggcttgaaaggtgtatggaaatggatgataaccaaaaaacctccaactgtctcagagtctcaggaaacaccagctggcaactcagagggtcttcctgacaaggttccatctccagaatccccagcatccataccagaaaaagagaaacccagctctccctcctctggcaaagggaaaactgagaaggcagagattcccatccttcctgacgtagagcagttttggcatgagagggacacagtcccttctgtacaggacaatgaccctatcccctgggaacatgaagatcaagagacaggggaaggtgttaaa

SEQ ID NO: 5 - DNA, 36 bp, Artificial Sequence
  primer
gtcgacatgctggcagctgcctttgcagactctaac

SEQ ID NO: 6 - DNA, 37 bp, Artificial Sequence
  primer
tactagtctatttaacaccttcccctgtctcttgatc

SEQ ID NO: 7 - DNA, 21 bp, Artificial Sequence
  primer
ctcctgctgttttctgcacct

SEQ ID NO: 8 - DNA, 22 bp, Artificial Sequence
  primer
agacaaaccagcctagatccca

SEQ ID NO: 9 - DNA, 28 bp, Artificial Sequence
  primer
tccgagctacggcgtactccaaaagtgt

(19) United States
(12) Patent Application Publication
(10) Pub. No.: US 2003/0191285 A1
(43) Pub. Date: Oct. 9, 2003

(54) Novel g protein-coupled receptor proteins and dnas thereof

(76) Inventors: Masanori Miwa, Ibaraki (JP); Yasushi Shintani, Osaka (JP); Hideki Matsui, Ibaraki (JP)

Correspondence Address: TAKEDA PHARMACEUTICALS NORTH AMERICA, INC, INTELLECTUAL PROPERTY DEPARTMENT, 475 HALF DAY ROAD, SUITE 500, LINCOLNSHIRE, IL, 60069, US

(21) Appl. No.: 10/203,131

(22) PCT Filed: Feb. 7, 2001
(86) PCT No.: PCT/JP01/00851

(30) Foreign Application Priority Data
Feb. 8, 2000 (JP) 2000-35680

(51) Int. Cl. C12Q 1/68; G01N 33/53; C07H 21/04; C12P 21/02; C12N 5/06; C07K 14/705; C07K 16/28

(52) U.S. Cl. 530/350; 530/388.22; 435/69.1; 435/320.1; 435/325; 536/23.5; 435/7.1; 435/6

(57) ABSTRACT

DNAs encoding human leukocyte-derived G protein-coupled receptor proteins or salts thereof are useful in: (1) determining ligands; (2) acquiring antibodies and antisera; (3) constructing a recombinant receptor protein expression system; (4) developing a receptor-bound assay system and screening candidate compounds for a drug using the expression system above; (5) designing drugs based on the comparison with ligand receptors having similar structures; (6) reagents in preparing probes, PCR primers, etc. for gene therapy; (7) constructing transgenic animals; (8) drugs such as gene preventives and remedies; etc.

### FIELD OF THE INVENTION

The present invention relates to novel human leukocyte-derived proteins or salts thereof and DNAs encoding the same, and the like.

### BACKGROUND ART

Many physiologically active substances like hormones, neurotransmitters, etc. regulate the functions of the body via specific receptor proteins present on cell membranes. Most of these receptor proteins are coupled to guanine nucleotide-binding proteins (hereinafter sometimes referred to as G proteins) to mediate the intracellular signal transduction through activation of the G proteins. These receptors possess a common structure comprising seven transmembrane domains and are thus referred to collectively as G protein-coupled receptor proteins or seven transmembrane receptors (7 TMR).

G protein-coupled receptor proteins exist on cells of a living body and each functional cell surface of cells and organs and play very important roles as the targets of molecules, for example, hormones, neurotransmitters, physiologically active substances and the like, which molecules regulate the functions of cells and organs in vivo. These receptors mediate signal transduction in a cell by binding to physiologically active substances and various reactions such as activation or inhibition of cells are induced.

To clarify the relationship between substances that regulate complicated biological functions in various cells and organs and their specific receptor proteins, in particular, G protein-coupled receptor proteins, would elucidate the functional mechanisms in various cells and organs in the body to provide a very important means for developing drugs closely associated with these functions.

For example, in various organs, their physiological functions are controlled in vivo through regulation by many hormones, hormone-like substances, neurotransmitters or physiologically active substances. In particular, physiologically active substances are found in numerous sites of the body and regulate the physiological functions through their corresponding receptor proteins. However, many unknown hormones, neurotransmitters or other physiologically active substances still exist in the body and, as to their receptor proteins, most of their structures have not yet been reported. Moreover, it is still unknown if there are subtypes of known receptor proteins.

It is also very important means for development of drugs to clarify the relationship between substances that regulate elaborate functions in vivo and their specific receptor proteins. Furthermore, in order to efficiently screen agonists and antagonists to receptor proteins for developing drugs, it is required to clarify the functions of receptor protein genes expressed in vivo and express the genes in an appropriate expression system.

In recent years, random analysis of cDNA sequences has been actively studied as a means for analyzing genes expressed in vivo. The sequences of cDNA fragments thus obtained have been registered on and published to databases as Expressed Sequence Tag (EST). However, since many ESTs contain sequence information only, it is difficult to predict their functions. Substances that inhibit the binding of G protein coupled proteins to physiological active substances (i.e., ligands) and substances that bind to physiologically active substances thereby to induce signal transduction similar to those induced by the physiologically active substances (i.e., ligands) have been used for pharmaceuticals as antagonists or agonists specific to the receptors for regulating the biological functions. Accordingly, it is very important to discover a new G protein-coupled receptor protein that is not only important for physiological expression in vivo but can be a target for developing pharmaceuticals and to clone the genes (e.g., cDNA), in search for a specific ligand, agonist, and antagonist of the novel G protein coupled receptor.

However, not all G protein coupled receptors have been found. Even now, there are many unknown G protein coupled receptors and those for which the corresponding ligands are unidentified, that is, orphan receptors. It has thus been seriously awaited to explore a novel G protein coupled receptor and clarify its function.

G Protein coupled receptors are useful in searching for novel physiologically active substances (i.e., ligands) using the signal transduction activity as an index and in searching for agonists and antagonists of the receptors. Even if no physiological ligand is found, agonists or antagonists of the receptors may be prepared by analyzing the physiological activities of the receptors through receptor inactivation experiments (knockout animal). Ligands, agonists, antagonists, etc. of these receptors are expected to be used as prophylactic/therapeutic drugs and diagnostics for diseases associated with dysfunction of the G protein coupled receptors.

Hypofunction or hyperfunction of G protein coupled receptors due to genetic mutation of the receptors in vivo often causes some disorders. In this case, the G protein coupled receptors may be used not only for administration of antagonists or agonists of the receptors, but also for gene therapy by introducing the receptor gene into the body (or some particular organ) or by introducing the antisense nucleic acid to the receptor gene. In such a gene therapy, information on the base sequence of the receptor gene is essentially required for searching any deletion or mutation in the gene. The receptor gene is also applicable as prophylactic/therapeutic drugs or diagnostics for diseases associated with dysfunction of the receptor.

### DISCLOSURE OF THE INVENTION

The present invention provides a novel and useful G protein-coupled receptor protein as described above. That is, the present invention provides a novel G protein-coupled receptor protein or its partial peptides, or salts thereof; polynucleotides (DNAs and RNAS, and derivatives thereof) containing polynucleotides (DNAs and RNAS, and derivatives thereof) encoding the G protein-coupled receptor protein or its partial peptides; recombinant vectors containing the polynucleotides; transformants bearing the recombinant vectors; methods for manufacturing the G protein-coupled receptor protein or salts thereof; antibodies to the G protein-coupled receptor protein or its partial peptides, or salts thereof; compounds that alter the expression level of said G protein-coupled receptor protein; methods for determination of ligands to the G protein-coupled receptor protein; methods for screening compounds (antagonists or agonists) or salts thereof that alter the binding property of ligands and the G protein-coupled receptor protein; kits for screening the same; compounds (antagonists and agonists) or salts thereof that alter the binding property of ligands obtainable by the screening method or obtainable using the screening kit and the G protein-coupled receptor protein; and pharmaceutical compositions comprising the compounds (antagonists or agonists) that alter the binding property of ligands to the G protein-coupled receptor protein, or compounds or salts thereof that alter the expression level of the G protein-coupled receptor protein; and the like.

The inventors performed extensive studies and as a result, succeeded in isolating cDNAs encoding a novel G protein-coupled receptor protein derived from human leukocyte, which resulted in successful analysis of the entire base sequence of the cDNAs. The amino acid sequence deduced from the base sequence has supported that the first to the seventh transmembrane domains were observed on the hydrophobic plotting analysis, confirming that the protein encoded by these cDNAs is a transmembrane G protein-coupled receptor protein passing through the membrane seven times. Based on these findings, the inventors have continued further studies and have come to accomplish the present invention.

Thus, the present invention relates to the following features:

(1) A protein containing the same or substantially the same amino acid sequence as the amino acid sequence represented by SEQ ID NO: 1, or a salt thereof;

(2) A partial peptide of the protein according to (1), or a salt thereof;

(3) The protein or its salt according to (1), wherein the same or substantially the same amino acid sequence as the amino acid sequence represented by SEQ ID NO: 1 is the amino acid sequence represented by SEQ ID NO: 3;

(4) A polynucleotide containing a polynucleotide encoding the protein according to (1);

(5) The polynucleotide according to (4), which is a DNA;

(6) The polynucleotide according to (4), which bears the base sequence represented by SEQ ID NO: 2 or SEQ ID NO: 4;

(7) A recombinant vector containing the polynucleotide according to (4);

(8) A transformant transformed by the recombinant vector according to (7);

(9) A method for manufacturing the protein or its salt according to (1), which comprises culturing the transformant according to (8) and producing the protein according to (1);

(10) An antibody to the protein according to (1) or the partial peptide according to (2), or a salt thereof;

(11) The antibody according to (10), which is a neutralizing antibody to inactivate signal transduction of the protein according to (1);

(12) A diagnostic composition comprising the antibody according to (10);

(13) A ligand to the protein or its salt according to (1), which is obtainable using the protein according to (1) or the partial peptide according to (2), or a salt thereof;

(14) A pharmaceutical composition comprising the ligand according to (13);

(15) A method for determining a ligand to the protein or its salt according to (1), which comprises using the protein according to (1) or the partial peptide according to (2), or a salt thereof;

(16) A method for screening a compound or a salt thereof that alters the binding property between a ligand and the protein or its salt according to (1), which comprises using the protein according to (1) or the partial peptide according to (2), or a salt thereof;

(17) A kit for screening a compound or a salt thereof that alters the binding property between a ligand and the protein or its salt according to (1), comprising the protein according to (1) or the partial peptide according to (2), or a salt thereof;

(18) A compound or a salt thereof that alters the binding property between a ligand and the protein or its salt according to (1), which is obtainable by the screening method according to (16) or using the screening kit according to (17);

(19) A pharmaceutical composition comprising a compound or a salt thereof that alters the binding property between a ligand and the protein or its salt according to (1), which is obtainable using the screening method according to (16) or the screening kit according to (17);

(20) A polynucleotide which is hybridizable to the polynucleotide according to (4) under high stringent conditions;

(21) A polynucleotide containing a complementary base sequence to the polynucleotide according to (4), or a part thereof;

(22) A method of quantifying mRNA of the protein according to (1), which comprises using the polynucleotide according to (4) or a part thereof;

(23) A method of quantifying the protein according to (1), which comprises using the antibody according to (10);

(24) A method for diagnosis of diseases associated with the function of the protein according to (1), which comprises using the method of quantifying according to (22) or (23);

(25) A method for screening a compound or a salt thereof that alters the expression level of the protein according to (1), which comprises using the method of quantifying according to (22);

(26) A method for screening a compound or a salt thereof that alters the amount of the protein according to (1) on cell membrane, which comprises using the method of quantifying according to (23);

(27) A compound or a salt thereof that alters the expression level of the protein according to (1), which is obtainable using the method of quantifying according to (25); and,

(28) A compound or a salt thereof that alters the amount of the protein according to (1) in a cell membrane, which is obtainable using the method of quantifying according to (26); etc.

The present invention further provides the following features.

(29) The protein or its salt according to (1) containing: (i) an amino acid sequence represented by SEQ ID NO: 1, an amino acid sequence represented by SEQ ID NO: 1, of which one, two, or more amino acids (preferably approximately 1 to 30 acids, more preferably approximately 1 to 9, and most preferably several (1 to 5)) amino acids are deleted; (ii) an amino acid sequence represented by SEQ ID NO: 1, to which one, two, or more amino acids (preferably approximately 1 to 30, more preferably approximately 1 to 10, and most preferably several (1 to 5)) amino acids are added; (iii) an amino acid sequence represented by SEQ ID NO: 1, in which one, two, or more amino acids (preferably approximately 1 to 30, more preferably approximately 1 to 10, and most preferably several (1 to 5)) amino acids are substituted by other amino acids; and (iv) a combination of the above amino acid sequences;

(30) A method of determining the ligand according to (15), which comprises contacting the protein or its salt according to (1) or the partial peptide or its salt according to (2) with a test compound;

(31) The method of determining the ligand according to (30), wherein the ligand is angiotensin, bombesin, canavinoid, cholecystokinin, glutamine, serotonin, melatonin, neuropeptide Y, opioid, purines, vasopressin, oxytocin, PACAP, secretin, glucagon, calcitonin, adrenomedulin, somatostatin, GHRH, CRF, ACTH, GRP, PTH, VIP (vasoactive intestinal polypeptide), somatostatin, dopamine, motilin, amylin, bradykinin, CGRP (calcitonin gene-related peptide), leukotrienes, pancreastatin, prostaglandins, thromboxane, adenosine, adrenaline, α and β-chemokines (e.g., IL-8, GROα, GROβ, GROγ, NAP-2, ENA-78, PF4, IP10, GCP-2, MCP-1, HC14, MCP-3, I-309, MIP-1α, MIP-1β, RANTES, etc.), endothelin, enterogastrin, histamine, neurotensin, TRH, pancreatic polypeptide or galanin;

(32) A method for screening according to (16), which comprises comparing (i) the case wherein the protein or its salt according to (1) or the partial peptide or its salt according to (2) are brought in contact with the ligand and (ii) the case wherein the protein or its salt according to (1) or the partial peptide or its salt according to (2) are brought in contact with the ligand and a test compound;

(33) A method for screening a compound or a salt thereof that alters the binding property between a ligand and the protein or its salt according to (1), which comprises measuring and comparing (i) an amount of labeled ligand bound to the protein or its salt according to (1) or the partial peptide or its salt according to (2) wherein the labeled ligand is brought in contact with the protein or its salt according to (1) or the partial peptide or its salt according to (2), and (ii) an amount of labeled ligand bound to the protein or its salt according to (1) or the partial peptide or its salt according to (2) wherein the labeled ligand and a test compound are brought in contact with the protein or its salt according to (1) or the partial peptide or its salt according to (2);

(34) A method for screening a compound or a salt thereof that alters the binding property between a ligand and the protein or its salt according to (1), which comprises measuring and comparing (i) an amount of labeled ligand bound to a cell containing the protein according to (1) when the labeled ligand is brought in contact with the cell, and (ii) an amount of labeled ligand bound to the cell when the labeled ligand and a test compound are brought in contact with the cell;

(35) A method for screening a compound or a salt thereof that alters the binding property between a ligand and the protein or its salt according to (1), which comprises measuring and comparing (i) an amount of labeled ligand bound to a membrane fraction of the cell containing the protein according to (1) when the labeled ligand is brought in contact with the cell membrane fraction, and (ii) an amount of labeled ligand bound to a membrane fraction of the cell when the labeled ligand and a test compound are brought in contact with the cell membrane fraction;

(36) A method for screening a compound or a salt thereof that alters the binding property between a ligand and the protein or its salt according to (1), which comprises measuring and comparing (i) an amount of labeled ligand bound to a protein expressed in a cell membrane of the transformant according to (8) by culturing the transformant, when the labeled ligand is brought in contact with the protein expressed, and (ii) an amount of labeled ligand bound to a protein expressed in cell membrane of the transformant according to (8) by culturing the transformant, when the labeled ligand and a test compound are brought in contact with the protein expressed;

(37) A method for screening a compound or a salt thereof that alters the binding property between a ligand and the protein or its salt according to (1), which comprises measuring and comparing (i) a protein-mediated cell stimulating activity when a compound that activates the protein or its salt according to (1) is brought in contact with a cell containing the protein according to (1), and (ii) a protein-mediated cell stimulating activity when a compound that activates the protein or its salt according to (1) and a test compound are brought in contact with a cell containing the protein according to (1);

(38) A method for screening a compound-or a salt thereof that alters the binding property between a ligand and the protein or its salt according to (1), which comprises measuring and comparing (i) a protein-mediated cell stimulating activity when a compound that activates the protein or its salt according to (1) is brought in contact with a protein expressed in a cell membrane of the transformant according to (8) by culturing the transformant, and (ii) a protein-mediated cell stimulating activity when a compound that activates the protein or its salt according to (1) and a test compound are brought in contact with a protein expressed in a cell membrane of the transformant according to (8) by culturing the transformant;

(39) The method for screening according to (37) or (38), wherein the compound that activates the protein according to (1) is angiotensin, bombesin, canavinoid, cholecystokinin, glutamine, serotonin, melatonin, neuropeptide Y, opioid, purines, vasopressin, oxytocin, PACAP, secretin, glucagon, calcitonin, adrenomedulin, somatostatin, GHRH, CRF, ACTH, GRP, PTH, VIP (vasoactive intestinal peptide), somatostatin, dopamine, motilin, amylin, bradykinin, CGRP (calcitonin gene-related peptide), leukotrienes, pancreastatin, prostaglandins, thromboxane, adenosine, adrenaline, a and β-chemokines (e.g., IL-8, GROα, GROβ, GROγ, NAP-2, ENA-78, PF4, IP10, GCP-2, MCP-1, HC14, MCP-3, I-309, MIP-1α, MIP-1β, RANTES, etc.), endothelin, enterogastrin, histamine, neurotensin, TRH, pancreatic polypeptide or galanin;

(40) A compound or a salt thereof that alters the binding property between a ligand and the protein or its salt according to (1), which is obtainable by the method for screening according to (32) through (39);

(41) A pharmaceutical composition comprising a compound or a salt thereof that alters the binding property between a ligand and the protein or its salt according to (1), which is obtainable by the method for screening according to (32) through (39);

(42) A kit for screening according to (17), comprising a cell containing the protein according to (1);

(43) A kit for screening according to (17), comprising a membrane fraction of a cell containing the protein according to (1);

(44) A kit for screening according to (17), comprising a protein expressed in a cell membrane of the transformant according to (8) by culturing the transformant;

(45) A compound or a salt thereof that alters the binding property between a ligand and the protein or its salt according to (1), which is obtainable using the screening kit according to (42) through (44);

(46) A pharmaceutical composition comprising a compound or a salt thereof that alters the binding property between a ligand and the protein or its salt according to (1), which is obtainable using the screening kit according to (42) through (44);

(47) A method for quantifying the protein according to (1), the partial peptide according to (2), or a salt thereof, which comprises contacting the antibody according to (10) the protein according to (1), the partial peptide according to (2), or a salt thereof;

(48) A method for quantifying the protein according to (1), the partial peptide according to (2), or a salt thereof, in a sample solution, which comprises competitively reacting the antibody according to (10) with a sample solution and the labeled protein or its salt according to (1) or the labeled partial peptide or its salt according to (2) and, measuring a ratio of the labeled protein or its salt according to (1) or the labeled partial peptide or its salt according to (2), which are bound to the antibody; and,

(49) A method for quantifying the protein according to (1), the partial peptide according to (2), or a salt thereof, in a sample solution, which comprises reacting a sample solution simultaneously or sequentially with antibody according to (10) immobilized on a carrier and the labeled antibody according to (10) and then measuring the activity of a labeling agent on the immobilized carrier; etc.

### BRIEF DESCRIPTION OF THE DRAWINGS

FIG. 1 shows the base sequence of DNA encoding human leukocyte-derived novel receptor protein hTGR2L of the present invention obtained in EXAMPLE 1, and the amino acid sequence deduced from the base sequence (continued to FIG. 2).

FIG. 2 shows the base sequence of DNA encoding human leukocyte-derived novel receptor protein hTGR2L of the present invention obtained in EXAMPLE 1, and the amino acid sequence deduced therefrom (continued from FIG. 1, continued to FIG. 3).

FIG. 3 shows the base sequence of DNA encoding human leukocyte-derived novel receptor protein hTGR2L of the present invention obtained in EXAMPLE 1, and the amino acid sequence deduced therefrom (continued from FIG. 3).

FIG. 4 shows the base sequence of DNA encoding human leukocyte-derived novel receptor protein hTGR2V of the present invention obtained in EXAMPLE 1, and the amino acid sequence deduced therefrom (continued to FIG. 5).

FIG. 5 shows the base sequence of DNA encoding human leukocyte-derived novel receptor protein hTGR2V of the present invention obtained in EXAMPLE 1, and the amino acid sequence deduced therefrom (continued from FIG. 4, continued to FIG. 5).

FIG. 6 shows the base sequence of DNA encoding human leukocyte-derived novel receptor protein hTGR2V of the present invention obtained in EXAMPLE 1, and the amino acid sequence deduced therefrom (continued from FIG. 5).

FIG. 7 shows the hydrophobic plotting of human leukocyte-derived novel receptor protein hTGR2L of the present invention prepared based on the amino acid sequence shown in FIGS. 1 through 3.

FIG. 8 shows the hydrophobic plotting of human leukocyte-derived novel receptor protein hTGR2V of the present invention prepared based on the amino acid sequence shown in FIGS. 1 through 3.

FIG. 9 shows the analytical results of expression distribution of hTGR2 in human tissues tested in EXAMPLE 3.

### BEST MODE OF EMBODIMENT OF THE INVENTION

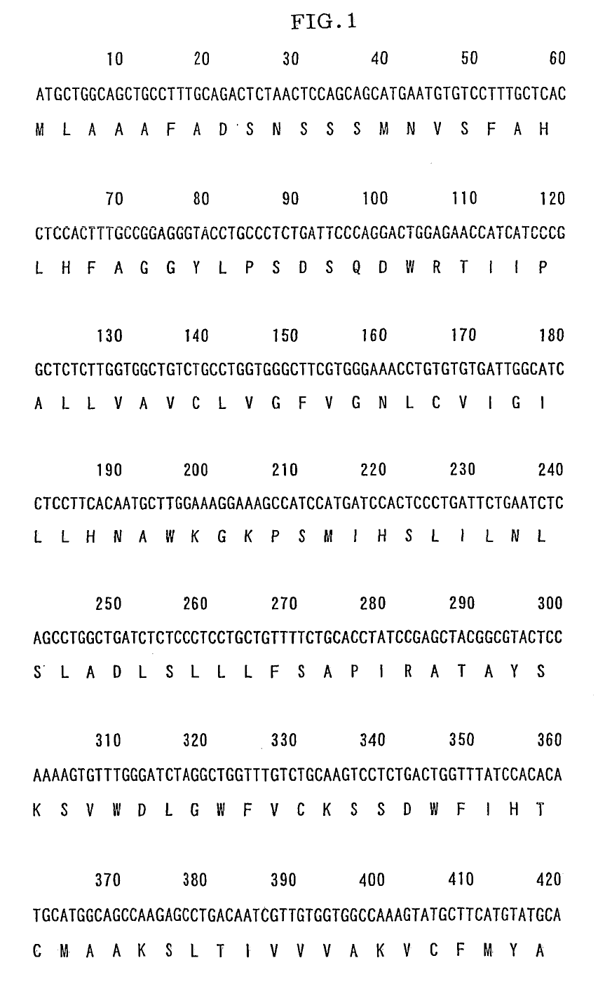

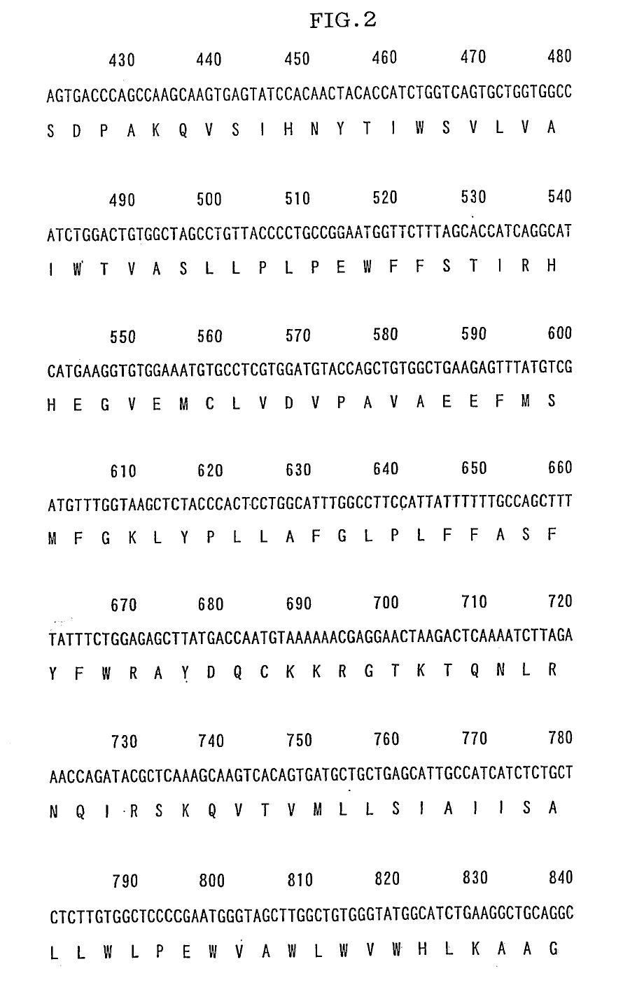

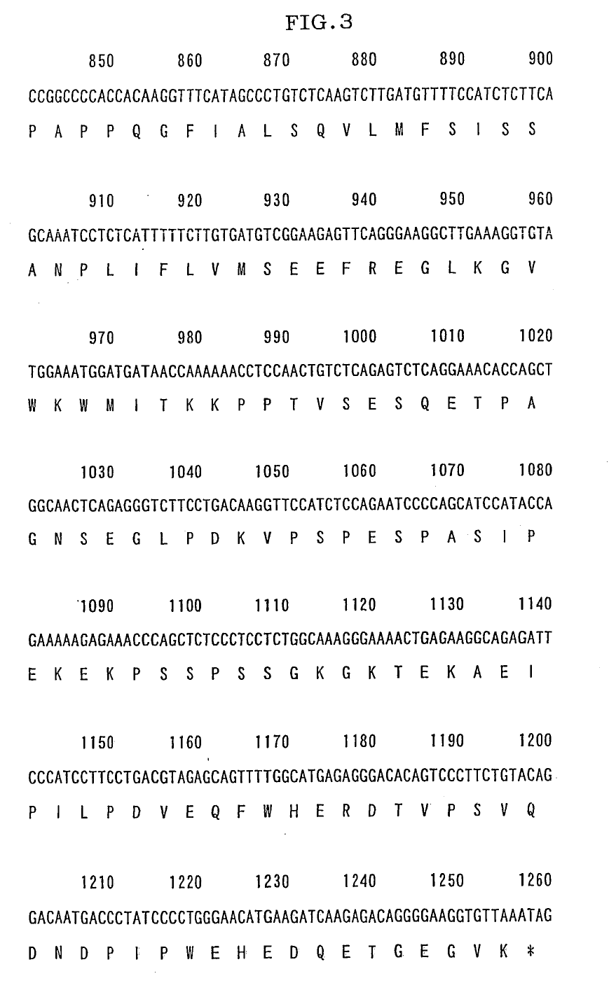

The G protein-coupled receptor protein (hereinafter sometimes referred to as the receptor protein) of the present invention is the receptor protein which has the same or substantially the same amino acid sequence as the amino acid sequence represented by SEQ ID NO: 1 (the amino acid sequence shown by FIGS. 1 through 3).

The receptor protein of the present invention may be any protein derived from any cells of, e.g., human and other mammal (e.g., guinea pig, rat, mouse, rabbit, swine, sheep, bovine, monkey, etc.) such as splenocyte, nerve cell, glial cell, β cell of pancreas, bone marrow cell, mesangial cell, Langerhans' cell, epidermic cell, epithelial cell, endothelial cell, fibroblast, fibrocyte, myocyte, fat cell, immune cell (e.g., macrophage, T cell, B cell, natural killer cell, mast cell, neutrophil, basophil, eosinophil, monocyte), megakaryocyte, synovial cell, chondrocyte, bone cell, osteoblast, osteoclast, mammary gland cell, hepatocyte or interstitial cell, or the corresponding precursor cells, stem cells, cancer cells, etc., or any tissues where such cells are present, for example, brain or any of brain regions (e.g., olfactory bulb, amygdaloid nucleus, basal ganglia, hippocampus, thalamus, hypothalamus, subthalamic nucleus, cerebral cortex, medulla oblongata, cerebellum, occipital lobes, frontal lobe, lateral lobe, putamen, caudate nucleus, corpus callosum, substantia nigra), spinal cord, pituitary gland, stomach, pancreas, kidneys, liver, gonads, thyroid gland, gallbladder, bone marrow, adrenal glands, skin, muscle, lung, digestive tract (e.g., large intestine, small intestine), vascular vessels, heart, thymus, spleen, submandibular gland, peripheral blood, peripheral blood cells, prostate, testes, orchis, ovaries, placenta, uterus, bones, joints, skeletal muscles, etc. (especially, the brain and each region of the brain). The receptor protein may also be synthetic protein.

The amino acid sequence which has substantially the same amino acid sequence as the amino acid sequence represented by SEQ ID NO: 1 includes an amino acid sequence having at least about 50% homology, preferably at least about 70% homology, more preferably at least about 80% homology, much more preferably at least about 90% homology, and most preferably at least about 95% homology, to the amino acid sequence represented by SEQ ID NO: 1.

Examples of the protein which has substantially the same amino acid sequence as the amino acid sequence shown by SEQ ID NO: 1 include a protein containing substantially the same amino acid sequence as the amino acid sequence represented by SEQ ID NO: 1 and having an activity substantially equivalent to that of the amino acid sequence represented by SEQ ID NO: 1, etc.

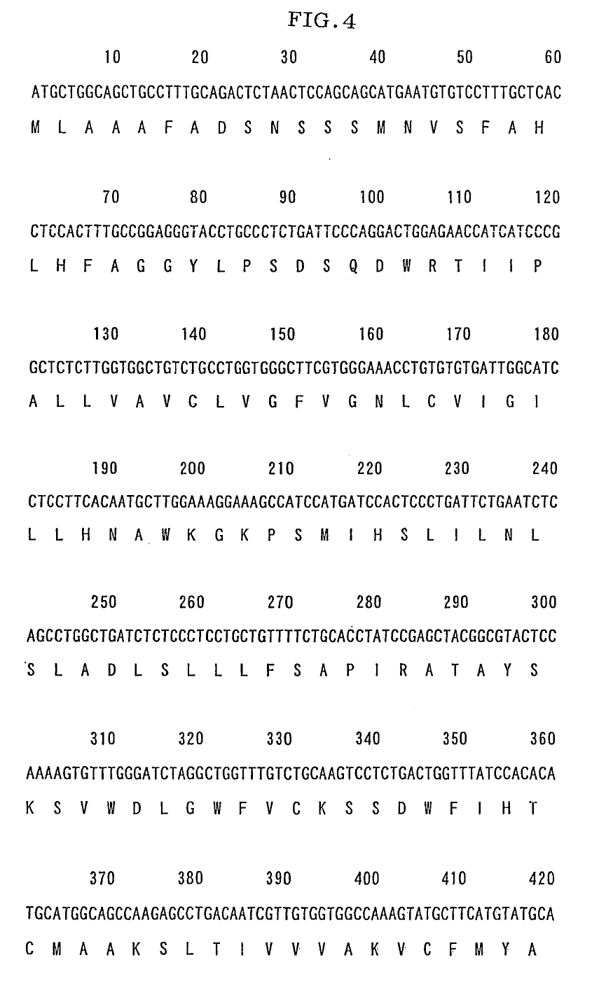

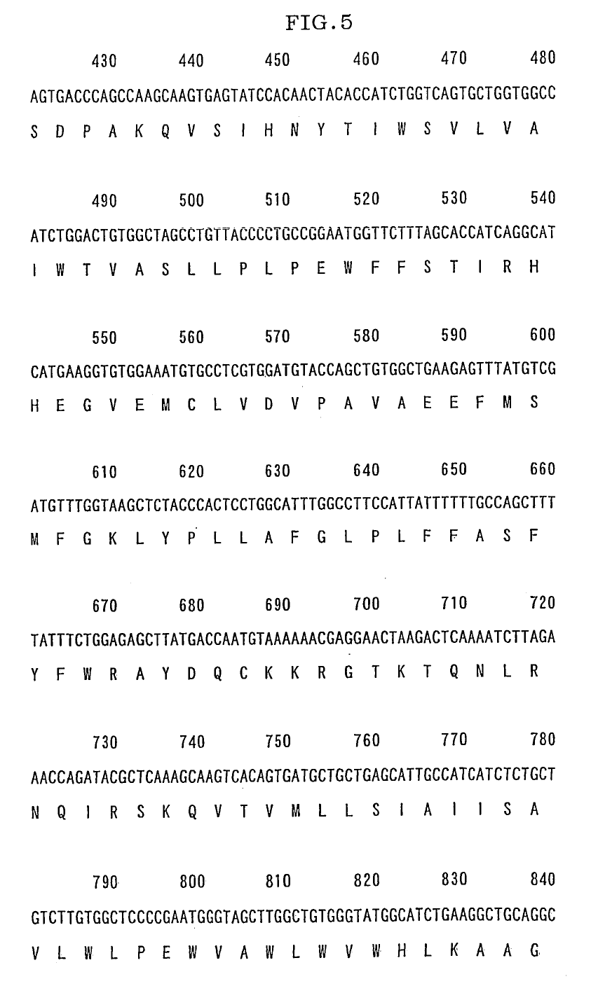

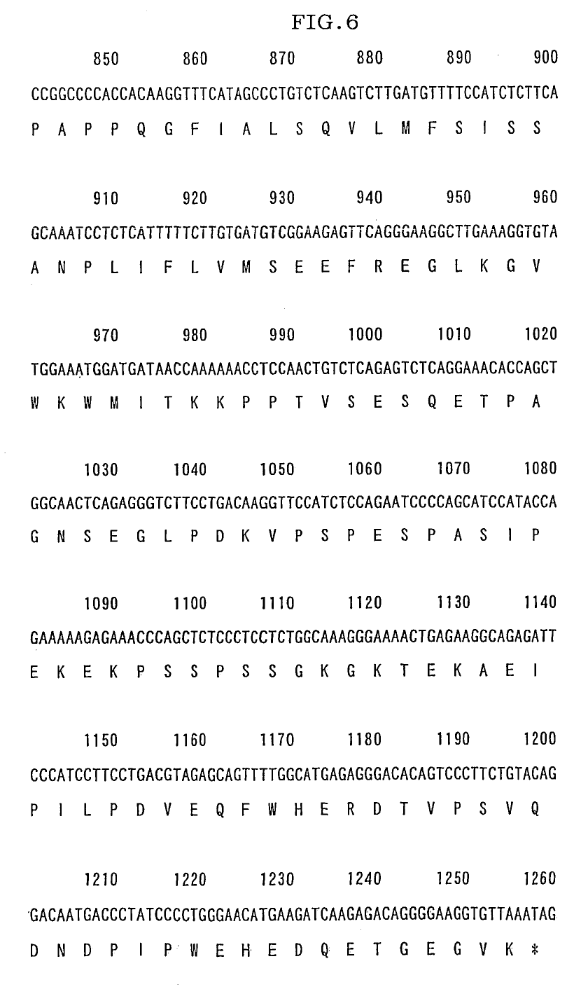

More specifically, examples of substantially the same amino acid sequence as the amino acid sequence represented by SEQ ID NO: 1 include the amino acid sequence represented by SEQ ID NO: 3 (amino acid sequence in FIGS. 4 to 6), etc.

Substantially the same activity includes, e.g., a ligand-binding property, a signal transduction activity, etc. Substantially the same means that these activities are the same in their nature. Therefore, it is preferred that the activities such as ligand-binding and signal transduction activities (e.g., about 0.01 to 100 times, preferably about 0.5 to 20 times, more preferably about 0.5 to 2 times) are equivalent, but it is allowable that differences among grades such as the level of these activities and molecular weight of the protein may be present.

The activities such as the ligand-binding property and signal transduction activity can be assayed by modifications of publicly known methods, but these activities may be measured by the ligand determination method and the screening method, which will be described later.

As the receptor proteins of the present invention, there are employed proteins containing (i) an amino acid sequence represented by SEQ ID NO: 1, of which one, two, or more amino acids (preferably approximately 1 to 30, more preferably approximately 1 to 10, and most preferably several (1 to 5)) amino acids are deleted; (ii) an amino acid sequence represented by SEQ ID NO: 1, to which one, two, or more amino acids (preferably approximately 1 to 30, more preferably approximately 1 to 10, and most preferably several (1 to 5)) amino acids are added; (iii) an amino acid sequence represented by SEQ ID NO: 1, in which one, two, or more amino acids (preferably approximately 1 to 30, more preferably approximately 1 to 10, and most preferably several (1 to 5)) amino acids are substituted by other amino acids; and (iv) a combination of the above amino acid sequences; and the like.

Throughout the present specification, the receptor proteins are represented in accordance with the conventional way of describing peptides, that is, the N-terminus (amino terminus) at the left hand and the C-terminus (carboxyl terminus) at the right hand. In the receptor proteins of the present invention including the receptor proteins containing the amino acid sequence shown by SEQ ID NO: 1, the C-terminus is usually in the form of a carboxyl group (—COOH) or a carboxylate (—COO⁻) but may be in the form of an amide (—CONH₂) or an ester (—COOR).

Herein, examples of the ester group shown by R include a C₁₋₆ alkyl group such as methyl, ethyl, n-propyl, isopropyl, n-butyl, etc.; a C₃₋₈ cycloalkyl group such as cyclopentyl, cyclohexyl, etc.; a C₆₋₁₂ aryl group such as phenyl, α-naphthyl, etc.; a C₇₋₁₄ aralkyl such as a phenyl-C₁₋₂ alkyl group, e.g., benzyl, phenethyl, etc.; an α-naphthyl-C₁₋₂ alkyl group such as α-naphthylmethyl, etc.; and the like. In addition, pivaloyloxymethyl or the like, which is used widely as an ester for oral administration, may also be used.

Where the receptor protein of the present invention contains a carboxyl group (or a carboxylate) at a position other than the C-terminus, it may be amidated or esterified and such an amide or ester is also included within the receptor protein of the present invention. For the ester group in this case, for example, the same groups as those described with respect to the above C-terminal and the like may be employed.

The receptor protein of the present invention further includes variants of the above receptor proteins wherein the amino group at the N-terminal methionine residue is protected with a protecting group (e.g., a C₁₋₆ acyl group such as a C₁₋₆ alkanoyl group e.g., formyl group, acetyl group, etc.); those wherein the N-terminal region is cleaved in vivo and the glutamyl group thus formed is pyroglutaminated; those wherein a substituent (e.g., —OH, —SH, amino group, imidazole group, indole group, guanidino group, etc.) on the side chain of an amino acid in the molecule is protected with a suitable protecting group (e.g., a C₁₋₆ acyl group such as a C₁₋₆ alkanoyl group , e.g., formyl group, acetyl group, etc.), or conjugated proteins such as glycoproteins having sugar chains.

Specific examples of the receptor protein of the present invention include a human-derived (more preferably human leukocyte-derived) receptor protein containing the amino acid sequence represented by SEQ ID NO: 1, a human-derived (more preferably human leukocyte-derived) receptor protein containing the amino acid sequence represented by SEQ ID NO: 3, etc.

The partial peptides of the receptor proteins (hereinafter sometimes referred to as the partial peptides) of the present invention may be any partial peptides so long as they are the partial peptides of the receptor proteins of the present invention described above. Among the receptor protein molecules of the present invention, partial peptides bearing the site exposed outside cell membranes and retaining substantially the same ligand binding activity, etc. may be employed.

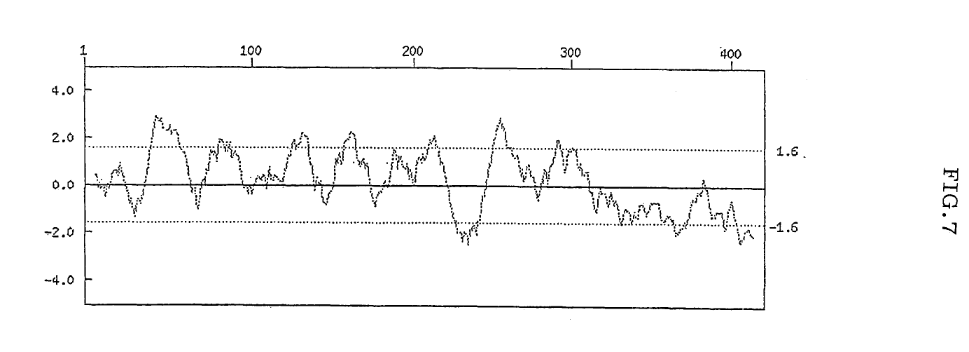

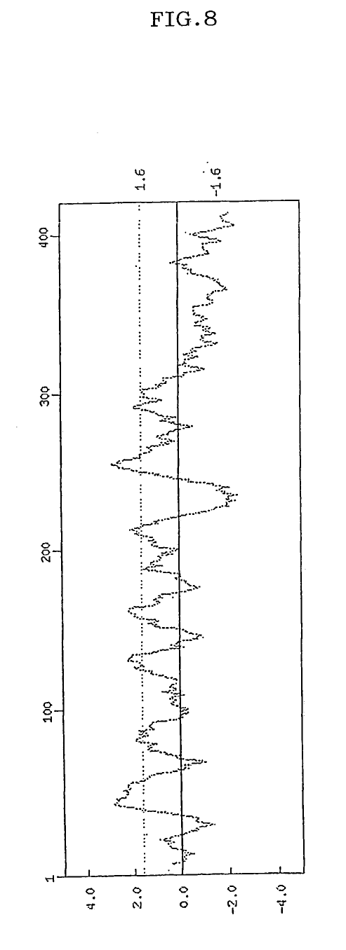

Specifically, the partial peptides of the receptor protein having the amino acid sequence represented by SEQ ID NO: 1 and the receptor protein having the amino acid sequence represented by SEQ ID NO: 3 are peptides containing the parts which have been analyzed to be extracellular domains (hydrophilic domains) in the hydrophobic plotting analysis shown by FIGS. 7 and 8, respectively. A peptide containing a hydrophobic domain part can be used as well. In addition, there may be employed a peptide containing each domain separately and a peptide containing plural domains together.

In the partial peptides of the present invention, the number of amino acids is at least 20, preferably at least 50 and more preferably at least 100, in the amino acid sequence which constitutes the receptor protein of the present invention.

Substantially the same amino acid sequence is used to mean an amino acid sequence having at least about 50% homology, preferably at least about 70% homology, more preferably at least about 80% homology, much more preferably at least about 90% homology, and most preferably at least about 95% homology, to these amino acid sequences.

Herein, the term “substantially equivalent ligand-binding activity” is intended to mean the same significance as defined above. The “substantially equivalent ligand-binding activity” can be assayed in the same way as described above.

Also, the receptor proteins of the present invention may be those having the amino acid sequences described above, of which one, two, or more amino acids (preferably approximately 1 to 10, and more preferably several (1 to 5)) amino acids are deleted; an amino acid sequence represented by SEQ ID NO: 1, to which one, two, or more amino acids (preferably approximately 1 to 20, more preferably approximately 1 to 10, and most preferably several (1 to 5)) amino acids are added; or, in which one, two, or more amino acids (preferably approximately 1 to 10, more preferably several, and most preferably approximately 1 to 5)) amino acids are substituted by other amino acids.

In the partial peptide of the present invention, the C-terminal is normally a carboxyl group (—COOH) or a carboxylate (—COO⁻) but the C-terminal may be an amide (—CONH₂) or an ester (—COOR), as has been described with the receptor protein of the present invention. Herein, R in the ester has the same significance as defined above. When the partial peptides of the present invention contain a carboxyl group (or a carboxylate) at the site other than the C-terminus, the carboxyl group may be amidated or esterified and those amides or esters are included in the partial peptides of the present invention. As the esters in this case, for example, the C-terminal esters described above may be employed.

As in the receptor protein of the present invention described above, the partial peptide of the present invention further includes those in which the amino group of the N-terminal methionine residue is protected by a protecting group, those in which the N-terminal residue is cleaved in vivo and the produced Gln is converted into pyroglutamate, those in which substituents on the side chains of amino acids in the molecule are protected by appropriate protecting groups, conjugated peptides such as those, to which sugar chains are bound, that is, glycopeptides, and the like.

As salts of the receptor protein of the present invention or partial peptide thereof, there are physiologically acceptable salts with acids or bases, preferably physiologically acceptable acid addition salts thereof. Examples of such salts are salts with inorganic acids (e.g., hydrochloric acid, phosphoric acid, hydrobromic acid, sulfuric acid), salts with organic acids (e.g., acetic acid, formic acid, propionic acid, fumaric acid, maleic acid, succinic acid, tartaric acid, citric acid, malic acid, oxalic acid, benzoic acid, methanesulfonic acid, benzenesulfonic acid), and the like.

The receptor protein of the present invention or salts thereof may be manufactured from the human or other mammalian cells or tissues described above in accordance with a publicly known method for purification of receptor proteins. Alternatively, the receptor protein of the present invention or salts thereof may also be manufactured by culturing a transformant transformed with a DNA encoding the receptor protein of the present invention, as will be later described. Furthermore, the receptor protein of the present invention or salts thereof may also be manufactured by the methods for synthesizing proteins, which will also be described hereinafter, or by modifications of these methods.

Where the receptor protein or salts thereof are manufactured from human or mammalian tissues or cells, human or mammalian tissues or cells are homogenized, then extracted with an acid or the like, and the extract is purified and isolated by a combination of chromatography techniques such as reverse phase chromatography, ion exchange chromatography, and the like.

To synthesize the receptor protein of the present invention or its partial peptide, or salts or amides thereof, commercially available resins that are normally used for protein synthesis may be used. Examples of such resins include chloromethyl resin, hydroxymethyl resin, benzhydrylamine resin, aminomethyl resin, 4-benzyloxybenzyl alcohol resin, 4-methylbenzhydrylamine resin, PAM resin, 4-hydroxymethylmethylphenyl acetamidomethyl resin, polyacrylamide resin, 4-(2′,4′-dimethoxyphenyl-hydroxymethyl)phenoxy resin, 4-(2′,4′-dimethoxyphenyl-Fmoc-aminoethyl)phenoxy resin, etc. Using such resins, amino acids in which a-amino groups and functional groups on the side chains are appropriately protected are condensed on the resin in the order of the amino acid sequence of the objective protein or peptide according to various condensation methods publicly known in the art. At the end of the reaction, the protein or peptide is excised from the resin and at the same time, the protecting groups are removed. Then, intramolecular disulfide bond-forming reaction is performed in a highly diluted solution to obtain the objective protein or partial peptide, or amides thereof.

For condensation of the protected amino acids described above, a variety of activation reagents for protein synthesis may be used, but carbodiimides are particularly preferable. Examples of such carbodiimides include DCC, N,N′-diisopropylcarbodiimide, N-ethyl-N′-(3-dimethylaminopropyl)carbodiimide, etc. For activation by these reagents, the protected amino acids in combination with a racemization inhibitor (e.g., HOBt, HOOBt) are added directly to the resin, or the protected amino acids are previously activated in the form of symmetric acid anhydrides, HOBt esters or HOOBt esters, followed by adding the thus activated protected amino acids to the resin.

Solvents suitable for use to activate the protected amino acids or condense with the resin may be chosen from solvents that are known to be usable for protein condensation reactions. Examples of such solvents are acid amides such as N,N-dimethylformamide, N,N-dimethylacetamide, N-methylpyrrolidone, etc.; halogenated hydrocarbons such as methylene chloride, chloroform, etc.; alcohols such as trifluoroethanol, etc.; sulfoxides such as dimethylsulfoxide, etc.; ethers such as pyridine, dioxane, tetrahydrofuran, etc.; nitrites such as acetonitrile, propionitrile, etc.; esters such as methyl acetate, ethyl acetate, etc.; and appropriate mixtures of these solvents. The reaction temperature is suitably chosen from the range known to be applicable to protein binding reactions and is usually selected in the range of approximately −20° C. to 50° C. The activated amino acid derivatives are used generally in an excess of 1.5 to 4 times. The condensation is examined using the ninhydrin reaction; when the condensation is insufficient, the condensation can be completed by repeating the condensation reaction without removal of the protecting groups. When the condensation is yet insufficient even after repeating the reaction, unreacted amino acids may be acetylated with acetic anhydride or acetylimidazole.

Examples of the protecting groups used to protect the starting amino groups include Z, Boc, tertiary pentyloxycarbonyl, isobornyloxycarbonyl, 4-methoxybenzyloxycarbonyl, Cl-Z, Br-Z, adamantyloxycarbonyl, trifluoroacetyl, phthaloyl, formyl, 2-nitrophenylsulphenyl, diphenylphosphinothioyl, Fmoc, etc.

A carboxyl group can be protected by, e.g., alkyl esterification (in the form of linear, branched or cyclic alkyl esters of the alkyl moiety such as methyl, ethyl, propyl, butyl, tertiary butyl, cyclopentyl, cyclohexyl, cycloheptyl, cyclooctyl, 2-adamantyl, etc.), aralkyl esterification (e.g., esterification in the form of benzyl ester, 4-nitrobenzyl ester, 4-methoxybenzyl ester, 4-chlorobenzyl ester, benzhydryl ester, etc.), phenacyl esterification, benzyloxycarbonyl hydrazidation, tertiary butoxycarbonyl hydrazidation, trityl hydrazidation, or the like.

The hydroxyl group of serine can be protected through, for example, its esterification or etherification. Examples of groups appropriately used for the esterification include a lower C₁₋₆ alkanoyl group, such as acetyl group, an aroyl group such as benzoyl group, and a group derived from carbonic acid such as benzyloxycarbonyl group and ethoxycarbonyl group. Examples of a group appropriately used for the etherification include benzyl group, tetrahydropyranyl group, t-butyl group, etc.

Examples of groups for protecting the phenolic hydroxyl group of tyrosine include Bzl, Cl₂-Bzl, 2-nitrobenzyl, Br-Z, tertiary butyl, etc.

Examples of groups used to protect the imidazole moiety of histidine include Tos, 4-methoxy-2,3,6-trimethylbenzenesulfonyl, DNP, benzyloxymethyl, Bum, Boc, Trt, Fmoc, etc.

Examples of the activated carboxyl groups in the starting amino acids include the corresponding acid anhydrides, azides, activated esters [esters of alcohols (e.g., pentachlorophenol, 2,4,5-trichlorophenol, 2,4-dinitrophenol, cyanomethyl alcohol, p-nitrophenol, HONB, N-hydroxysuccimide, N-hydroxyphthalimide, HOBt)], etc. As the activated amino acids in which the amino groups are activated in the starting material, the corresponding phosphoric amides are employed.

To eliminate (split off) the protecting groups, there are used catalytic reduction under hydrogen gas flow in the presence of a catalyst such as Pd-black or Pd-carbon; an acid treatment with anhydrous hydrogen fluoride, methanesulfonic acid, trifluoromethane-sulfonic acid or trifluoroacetic acid, or a mixture solution of these acids; a treatment with a base such as diisopropylethylamine, triethylamine, piperidine, piperazine, or the like; and reduction with sodium in liquid ammonia, etc. The elimination of the protecting group by the acid treatment described above is carried out generally at a temperature of approximately −20° C. to 40° C. In the acid treatment, it is efficient to add a cation scavenger such as anisole, phenol, thioanisole, m-cresol, p-cresol, dimethylsulfide, 1,4-butanedithiol, 1,2-ethanedithiol, etc. Furthermore, 2,4-dinitrophenyl group known as the protecting group for the imidazole of histidine is removed by a treatment with thiophenol. Formyl group used as the protecting group of the indole of tryptophan is eliminated by the aforesaid acid treatment in the presence of 1,2-ethanedithiol or 1,4-butanedithiol, as well as by a treatment with an alkali such as a dilute sodium hydroxide solution, dilute ammonia, etc.

Protection of functional groups that should not take part in the reaction of the starting materials, protecting groups, elimination of the protecting groups and activation of functional groups involved in the reaction may be appropriately selected from publicly known groups and publicly known means.

In another method for obtaining the amides of the protein, for example, the α-carboxyl group of the carboxy terminal amino acid is first protected by amidation; the peptide (protein) chain is then extended from the amino group side to a desired length. Thereafter, a protein, in which only the protecting group of the N-terminal α-amino group in the peptide chain has been eliminated from the protein, and a protein, in which only the protecting group of the C-terminal carboxyl group has been eliminated, are manufactured. The two proteins are condensed in a mixture of the solvents described above. The details of the condensation reaction are the same as described above. After the protected protein obtained by the condensation is purified, all the protecting groups are eliminated by the method described above to give the desired crude protein. This crude protein is purified by various known purification means. Lyophilization of the major fraction gives the amide of the desired protein.

To prepare the esterified protein, for example, the α-carboxyl group of the carboxy terminal amino acid is condensed with a desired alcohol to prepare the amino acid ester, which is followed by procedure similar to the preparation of the amidated protein above to give the desired esterified protein.

The partial peptide of the receptor protein of the present invention or salts thereof can be manufactured by publicly known methods for peptide synthesis, or by cleaving the receptor protein of the present invention with an appropriate peptidase. For the methods for peptide synthesis, for example, either solid phase synthesis or liquid phase synthesis may be used. That is, the partial peptide or amino acids that can construct the receptor protein of the present invention are condensed with the remaining part of the partial peptide. Where the product contains protecting groups, these protecting groups are removed to give the desired peptide. Publicly known methods for condensation and elimination of the protecting groups are described in 1)-5) below.

1) M. Bodanszky & M. A. Ondetti: Peptide Synthesis, Interscience Publishers, New York (1966)

2) Schroeder & Luebke: The Peptide, Academic Press, New York (1965)

3) Nobuo Izumiya, et al.: Peptide Gosei-no-Kiso to Jikken (Basics and experiments of peptide synthesis), published by Maruzen Co. (1975)

4) Haruaki Yajima & Shunpei Sakakibara: Seikagaku Jikken Koza (Biochemical Experiment) 1, Tanpakushitsu no Kagaku (Chemistry of Proteins) IV, 205 (1977)

5) Haruaki Yajima ed.: Zoku lyakuhin no Kaihatsu (A sequel to Development of Pharmaceuticals), Vol. 14, Peptide Synthesis, published by Hirokawa Shoten

After completion of the reaction, the product may be purified and isolated by a combination of conventional purification methods such as solvent extraction, distillation, column chromatography, liquid chromatography and recrystallization to give the partial peptide of the present invention. When the partial peptide obtained by the above methods is in a free form, the peptide can be converted into an appropriate salt by a publicly known method; when the peptide is obtained in a salt form, it can be converted into a free form by a publicly known method.

The polynucleotide encoding the receptor protein of the present invention may be any polynucleotide so long as it contains the base sequence (DNA or RNA, preferably DNA) encoding the receptor protein of the present invention described above. Such a polynucleotide includes DNA and RNA including mRNA that encodes the receptor protein of the present invention. The polynucleotide may be either double-stranded or single-stranded. When the polynucleotide is double-stranded, it may be double-stranded DNA, double-stranded RNA or a DNA:RNA hybrid. When the polynucleotide is single-stranded, it may be a sense strand (i.e., coding strand) or an antisense strand (i.e., non-coding strand).

Using the polynucleotide encoding the receptor protein of the present invention, mRNA of the receptor protein of the present invention can be quantified by, for example, the method publicly known by the extra number of Jikken Igaku (Experimental Medical Science), 15(7), “New PCR and its application” (1997) or by its modified method.

The DNA encoding the receptor protein of the present invention may be any one of genomic DNA, genomic DNA library, cDNA derived from the cells or tissues described above, cDNA library derived from the cells or tissues described above and synthetic DNA. The vector to be used for the library may be any of bacteriophage, plasmid, cosmid, phagemid and the like. In addition, the DNA can be directly amplified by reverse transcriptase polymerase chain reaction (hereinafter abbreviated as RT-PCR) with the total RNA or mRNA fraction prepared from the cells or tissues described above.

Specifically, the DNA encoding the receptor protein of the present invention may be any DNA, as long as it is a DNA containing the base sequence represented by, e.g., SEQ ID NO: 2 or SEQ ID NO: 4, or a DNA having a base sequence hybridizable to the base sequence represented by SEQ ID NO: 2 or SEQ ID NO: 4 under high stringent conditions and encoding a receptor protein which has the activities substantially equivalent to those of the receptor protein of the present invention (e.g., a ligand binding activity, a signal transduction activity, etc.).

Examples of the DNA that is hybridizable to the base sequence represented by SEQ ID NO: 2 or SEQ ID NO: 4 include a DNA having at least about 70% homology, preferably at least about 80% homology, more preferably at least about 90% homology, most preferably at least about 95% homology, to the amino acid sequence represented by SEQ ID NO: 2 or SEQ ID NO: 4; and the like.

The hybridization can be carried out by publicly known methods or by a modification thereof, for example, according to the method described in Molecular Cloning, 2nd Ed., J. Sambrook et al., Cold Spring Harbor Lab. Press, (1989). A commercially available library may also be used according to the instructions of the attached manufacturer's protocol. The hybridization can be carried out preferably under high stringent conditions.

The high stringent conditions used herein are, for example, those in a sodium concentration at about 19 to about 40 mM, preferably about 19 to about 20 mM at a temperature of about 50 to about 70° C., preferably about 60 to about 65° C. In particular, hybridization conditions in a sodium concentration at about 19 mM at a temperature of about 65° C. are most preferred.

More specifically, for the DNA encoding the receptor protein having the amino acid sequence represented by SEQ ID NO: 1, there may be employed a DNA having the base sequence represented by SEQ ID NO: 2 and, a DNA having the base sequence represented by SEQ ID NO: 4 may be used as the DNA encoding the receptor protein having the amino acid sequence represented by SEQ ID NO: 4.

The polynucleotide comprising a part of the base sequence of a DNA encoding the receptor protein of the present invention or a part of the base sequence complementary to the DNA is intended to include not only a DNA encoding the partial peptide of the present invention described below but also an RNA.

According to the present invention, antisense polynucleotides (nucleic acids) that can inhibit replication or expression of the G protein-coupled receptor protein gene can be designed and synthesized based on the cloned or determined base sequence information of the DNA encoding the G protein-coupled receptor protein. Such polynucleotides (nucleic acids) can hybridize to the RNA of the G protein-coupled receptor protein gene and inhibit the synthesis or function of the RNA, or can regulate/control the expression of the G protein-coupled receptor protein gene via the interaction with RNAs associated with the G protein-coupled receptor protein. Polynucleotides complementary to the specified sequences of RNAs associated with the G protein-coupled receptor protein and polynucleotides that can specifically hybridize to RNAs associated with the G protein-coupled receptor protein are useful for regulating/controlling the expression of the G protein-coupled receptor protein gene in vivo and in vitro. These polynucleotides are also useful for the treatment and diagnosis of diseases. The term “correspond” is used to mean homologous or complementary to a specific sequence of nucleotides including the gene, base sequences or nucleic acids. As between nucleotides, base sequences or nucleic acids and peptides (proteins), the term “corresponding” usually refers to amino acids of a peptide (protein) that is instructed to be derived from the sequence of nucleotides (nucleic acids) or its complements. The 5′ end hairpin loop, 5′ end 6-base-pair repeats, 5′ end untranslated region, polypeptide translation initiation codon, protein coding region, ORF translation initiation codon, 3′ untranslated region, 3′ end palindrome region, and 3′ end hairpin loop of the G protein-coupled receptor protein gene may be selected as preferred target regions, though any region may be a target within the G protein-coupled receptor protein genes.

The relationship between the targeted nucleic acids and the polynucleotides complementary to at least a portion of the target region, specifically the relationship between the target and the polynucleotides hybridizable to the target, is denoted to be in “an antisense”. The antisense polynucleotides may be polydeoxynucleotides containing 2-deoxy-D-ribose, polydeoxynucleotides containing D-ribose, any other type of polynucleotides which are N-glycosides of a purine or pyrimidine base, other polymers containing non-nucleotide backbones (e.g., protein nucleic acids and synthetic sequence-specific nucleic acid polymers that are commercially available), other polymers containing nonstandard linkages (provided that the polymers contain nucleotides with such a configuration that allows base pairing or base stacking, as is found in DNAs or RNAs), etc. The antisense polynucleotides may be a double-stranded DNA, a single-stranded DNA, a double-stranded RNA, a single-stranded RNA and also a DNA:RNA hybrid, and further includes unmodified polynucleotides (or unmodified oligonucleotides), those with publicly known types of modifications, for example, those with labels known in the art, those with caps, methylated polynucleotides, those with substitution of one or more naturally occurring nucleotides with their analogue, those with intramolecular modifications of nucleotides such as those with uncharged linkages (e.g., methyl phosphonates, phosphotriesters, phosphoramidates, carbamates, etc.) and those with charged linkages or sulfur-containing linkages (e.g., phosphorothioates, phosphorodithioates, etc.), those having side chain groups such as proteins (including nucleases, nuclease inhibitors, toxins, antibodies, signal peptides, poly-L-lysine, etc.), saccharides (e.g., monosaccharides, etc.), etc., those with intercalators (e.g., acridine, psoralen, etc.), those containing chelators (e.g., metals, radioactive metals, boron, oxidative metals, etc.), those containing alkylating agents, those with modified linkages (e.g., a anomeric nucleic acids, etc.). Herein the terms “nucleoside”, “nucleotide” and “nucleic acid” are used to refer to moieties that contain not only the purine and pyrimidine bases, but also other heterocyclic bases, which have been modified. Such modifications include methylated purines and pyrimidines, acylated purines and pyrimidines and other heterocyclic rings. Modified nucleotides and modified nucleotides also include modifications on the sugar moiety, for example, wherein one or more hydroxyl groups may optionally be replaced with a halogen, aliphatic groups, or may be converted into the corresponding functional groups such as ethers, amines, or the like.

The antisense polynucleotide (nucleic acid) of the present invention is RNA, DNA or a modified nucleic acid (RNA, DNA). Specific examples of the modified nucleic acid are, but not limited to, sulfurized and thiophosphate derivatives of nucleic acids and those resistant to degradation of polynucleoside or oligonucleoside amides. The antisense nucleic acids of the present invention can be modified preferably based on the following design, that is, by increasing the intracellular stability of the antisense nucleic acid, increasing the cellular permeability of the antisense nucleic acid, increasing the affinity of the nucleic acid to the target sense strand to a higher level, or minimizing the toxicity, if any, of the antisense nucleic acid.

Many such modifications are known in the art, as disclosed in J. Kawakami, et al., Pharm. Tech. Japan, Vol. 8, pp.247, 1992; Vol. 8, pp.395, 1992; S. T. Crooke et al. ed., Antisense Research and Applications, CRC Press, 1993; etc.

The antisense nucleic acid of the present invention may contain altered or modified sugars, bases or linkages. The antisense nucleic acid may also be provided in a specialized form such as liposomes, microspheres or may be applied to gene therapy or may be provided in combination with attached moieties. Such attached moieties include polycations such as polylysine that act as charge neutralizers of the phosphate backbone, or hydrophobic moieties such as lipids (e.g., phospholipids, cholesterols, etc.) that enhance the interaction with cell membranes or increase uptake of the nucleic acid. Preferred examples of the lipids to be attached are cholesterols or derivatives thereof (e.g., cholesteryl chloroformate, cholic acid, etc.). These moieties may be attached at the 3′ or 5′ ends of the nucleic acid and may be also attached through a base, sugar, or intramolecular nucleoside linkage. Other moieties may be capping groups specifically placed at the 3′ or 5′ ends of the nucleic acid to prevent degradation by nuclease such as exonuclease, RNase, etc. Such capping groups include, but are not limited to, hydroxyl protecting groups known in the art, including glycols such as polyethylene glycol, tetraethylene glycol, and the like. The inhibitory activity of the antisense nucleic acid can be examined using the transformant of the present invention, the gene expression system of the present invention in vitro or in vivo, or the translation system of the G protein-coupled receptor protein of the present invention in vitro and in vivo. The nucleic acid can be applied to cells by a variety of publicly known methods.

The DNA encoding the partial peptide of the present invention may be any DNA, so long as it contains the base sequence encoding the partial peptide of the present invention described above. The DNA may also be any of genomic DNA, genomic DNA library, cDNA derived from the cells and tissues described above, cDNA library derived from the cells and tissues described above and synthetic DNA. The vector to be used for the library may be any of bacteriophage, plasmid, cosmid, phagemid and the like. In addition, the DNA can be directly amplified by reverse transcriptase polymerase chain reaction (hereinafter abbreviated as RT-PCR) with the total RNA or mRNA fraction prepared from the cells or tissues described above.

Specifically, as the DNA encoding the partial peptide of the present invention there are employed, for example, (1) a DNA that has a part of the base sequence of the DNA containing the base sequence represented by SEQ ID NO: 2 or SEQ ID NO: 4, or (2) a DNA having a base sequence hybridizable to the base sequence represented by SEQ ID NO: 2 or SEQ ID NO: 4 under high stringent conditions and containing a part of the base sequence of the DNA encoding a receptor protein having substantially the same activities (i.e., a ligand binding activity, a signal transduction activity and the like) as those of the receptor protein of the present invention.

Examples of the DNA that is hybridizable to the base sequence represented by SEQ ID NO: 2 or SEQ ID NO: 4 include a DNA containing the base sequence having at least about 70% homology, preferably at least about 80% homology, more preferably at least about 90% homology, most preferably at least about 95% homology, to the base sequence represented by SEQ ID NO: 2 or SEQ ID NO: 4.

For cloning of the DNA that completely encodes the receptor protein or its partial peptide of the present invention (hereinafter sometimes referred to as the receptor protein of the present invention), the DNA may be either amplified by PCR using synthetic DNA primers containing a part of the base sequence of the receptor protein of the present invention, or the DNA inserted into an appropriate vector can be screened by hybridization with a labeled DNA fragment or synthetic DNA that encodes a part or entire region of the receptor protein of the present invention. The hybridization can be carried out, for example, according to the method described in Molecular Cloning, 2nd (J. Sambrook et al., Cold Spring Harbor Lab. Press, 1989). The hybridization may also be performed using commercially available library in accordance with the protocol described in the attached instructions.

Conversion of the base sequence of DNA can be carried out according to publicly known methods such as the ODA-LA PCR method, the Gupped duplex method, the Kunkel method, etc., or its modifications, using a publicly known kit available as Mutan™-super Express Km (manufactured by Takara Shuzo Co., Ltd.), Mutan™-K (manufactured by Takara Shuzo Co., Ltd.), etc.

The cloned DNA encoding the receptor protein of the present invention can be used as it is, depending upon purpose or, if desired, after digestion with a restriction enzyme or after addition of a linker thereto. The DNA may contain ATG as a translation initiation codon at the 5′ end thereof and TAA, TGA or TAG as a translation termination codon at the 3′ end thereof. These translation initiation and termination codons may also be added by using an appropriate synthetic DNA adapter.

The expression vector of the receptor protein of the present invention can be manufactured, for example, by (a) excising the desired DNA fragment from the DNA encoding the receptor protein of the present invention, (b) followed by ligation of the DNA fragment with an appropriate expression vector downstream a promoter in the vector.

Examples of the vector include plasmids derived form E. coli (e.g., pBR322, pBR325, pUC12, pUC13), plasmids derived from Bacillus subtilis (e.g., pUB110, pTP5, pC194), plasmids derived from yeast (e.g., pSH19, pSH15), bacteriophages such as λ phage, etc., animal viruses such as retrovirus, vaccinia virus, baculovirus, etc. as well as pA1-11, pXT1, pRc/CMV, pRc/RSV, pcDNAI/Neo, etc.

The promoter used in the present invention may be any promoter if it matches well with a host to be used for gene expression. In the case of using animal cells as the host, examples of the promoter include SRα promoter, SV40 promoter, HIV·LTR promoter, CMV promoter, HSV-TK promoter, etc.

Among them, CMV (cytomegalovirus) promoter, SRα promoter or the like is preferably used. Where the host is bacteria of the genus Escherichia, preferred examples of the promoter include trp promoter, lac promoter, recA promoter, λP_{L} promoter, lpp promoter, T7 promoter, etc. In the case of using bacteria of the genus Bacillus as the host, preferred example of the promoter are SPO1 promoter, SPO2 promoter, penP promoter, etc. When yeast is used as the host, preferred examples of the promoter are PHO5 promoter, PGK promoter, GAP promoter, ADH promoter, etc. When insect cells are used as the host, preferred examples of the promoter include polyhedrin prompter, P10 promoter, etc.

In addition to the foregoing examples, the expression vector may further optionally contain an enhancer, a splicing signal, a poly A addition signal, a selection marker, SV40 replication origin (hereinafter sometimes abbreviated as SV40ori), etc. Examples of the selection marker include dihydrofolate reductase (hereinafter sometimes abbreviated as dhfr) gene [methotrexate (MTX) resistance], ampicillin resistant gene (hereinafter sometimes abbreviated as Amp^{r}), neomycin resistant gene (hereinafter sometimes abbreviated as Neo^{r}, G418 resistance), etc. In particular, when dhfr gene is used as the selection marker together with CHO (dhfr⁻) cell, selection can also be made on thymidine free media.

If necessary, a signal sequence that matches with a host is added to the N-terminal side of the receptor protein of the present invention. Examples of the signal sequence that can be used are Pho A signal sequence, OmpA signal sequence, etc. in the case of using bacteria of the genus Escherichia as the host; α-amylase signal sequence, subtilisin signal sequence, etc. in the case of using bacteria of the genus Bacillus as the host; MFα signal sequence, SUC2 signal sequence, etc. in the case of using yeast as the host; and insulin signal sequence, α-interferon signal sequence, antibody molecule signal sequence, etc. in the case of using animal cells as the host, respectively.

Using the vector containing the DNA encoding the receptor protein of the present invention thus constructed, transformants can be manufactured.

Examples of the host, which may be employed, are bacteria belonging to the genus Escherichia, bacteria belonging to the genus Bacillus, yeast, insect cells, insects, animal cells, and the like.

Specific examples of the bacteria belonging to the genus Escherichia include Escherichia coli K12 DH1 [Proc. Natl. Acad. Sci. U.S.A., 60, 160 (1968)], JM103 [Nucleic Acids Research, 9, 309 (1981)], JA221 [Journal of Molecular Biology, 120, 517 (1978)], HB101 [Journal of Molecular Biology, 41, 459 (1969)], C600 [Genetics, 39, 440 (1954)], etc.

Examples of the bacteria belonging to the genus Bacillus include Bacillus subtilis MI114 [Gene, 24, 255 (1983)], 207-21 [Journal of Biochemistry, 95, 87 (1984)], etc.

Examples of yeast include Saccharomiyces cereviseae AH22, AH22R⁻, NA87-11A, DKD-5D, 20B-12, Schizosaccharomyces pombe NCYC1913, NCYC2036, Pichia pastoris KM71, etc.

Examples of insect cells include, for the virus AcNPV, Spodoptera frugiperda cell (Sf cell), MG1 cell derived from mid-intestine of Trichoplusia ni, High Five™ cell derived from egg of Trichoplusia ni, cells derived from Mamestra brassicae, cells derived from Estigmena acrea, etc.; and for the virus BmNPV, Bombyx mori N cell (BmN cell), etc. is used. Examples of the Sf cell which can be used are Sf9 cell (ATCC CRL1711) and Sf21 cell (both cells are described in Vaughn, J. L. et al., In vivo, 13, 213-217 (1977).

As the insect, for example, a larva of Bombyx mori can be used (Maeda et al., Nature, 315, 592 (1985)).

Examples of animal cells include monkey cell COS-7, Vero, Chinese hamster cell CHO (hereinafter referred to as CHO cell), dhfr gene deficient Chinese hamster cell CHO (hereinafter simply referred to as CHO(dhfr⁻) cell), mouse L cell, mouse AtT-20, mouse myeloma cell, rat GH 3, human FL cell, etc.

Bacteria belonging to the genus Escherichia can be transformed, for example, by the method described in Proc. Natl. Acad. Sci. U.S.A., 69, 2110 (1972), Gene, 17, 107 (1982), etc.

Bacteria belonging to the genus Bacillus can be transformed, for example, by the method described in Molecular & General Genetics, 168, 111 (1979), etc.

Yeast can be transformed, for example, by the method described in Methods in Enzymology, 194, 182-187 (1991), Proc. Natl. Acad. Sci. U.S.A., 75, 1929 (1978), etc.

Insect cells or insects can be transformed, for example, according to the method described in Bio/Technology, 6, 47-55(1988), etc.

Animal cells can be transformed, for example, according to the method described in Saibo Kogaku (Cell Engineering), extra issue 8, Shin Saibo Kogaku Jikken Protocol (New Cell Engineering Experimental Protocol), 263-267 (1995), published by Shujunsha, or Virology, 52, 456 (1973).

Thus, the transformant transformed with the expression vector containing the DNA encoding the receptor protein can be obtained.

Where the host is bacteria belonging to the genus Escherichia or the genus Bacillus, the transformant can be appropriately cultured in a liquid medium which contains materials required for growth of the transformant such as carbon sources, nitrogen sources, inorganic materials, etc. Examples of the carbon sources include glucose, dextrin, soluble starch, sucrose, etc. Examples of the nitrogen sources include inorganic or organic materials such as ammonium salts, nitrate salts, corn steep liquor, peptone, casein, meat extract, soybean cake, potato extract, etc. Examples of the inorganic materials are calcium chloride, sodium dihydrogenphosphate, magnesium chloride, etc. In addition, yeast, vitamins, growth promoting factors etc. may also be added to the medium. Preferably, pH of the medium is adjusted to about 5 to about 8.

A preferred example of the medium for culturing the bacteria belonging to the genus Escherichia is M9 medium supplemented with glucose and Casamino acids [Miller, Journal of Experiments in Molecular Genetics, 431-433, Cold Spring Harbor Laboratory, New York, 1972]. If necessary, a chemical such as 3β-indolylacrylic acid can be added to the medium thereby to activate the promoter efficiently. Where the bacteria belonging to the genus Escherichia are used as the host, the transformant is usually cultivated at about 15° C. to about 43° C. for about 3 hours to about 24 hours. If necessary, the culture may further be aerated or agitated.

Where the bacteria belonging to the genus Bacillus are used as the host, the transformant is cultivated generally at about 30° C. to about 40° C. for about 6 hours to about 24 hours. If necessary, the culture can be aerated or agitated.

Where yeast is used as the host, the transformant is cultivated, for example, in Burkholder's minimal medium [Bostian, K. L. et al., Proc. Natl. Acad. Sci. U.S.A., 77, 4505 (1980)] or in SD medium supplemented with 0.5% Casamino acids [Bitter, G. A. et al., Proc. Natl. Acad. Sci. U.S.A., 81, 5330 (1984)]. Preferably, pH of the medium is adjusted to about 5 to about 8. In general, the transformant is cultivated at about 20° C. to about 35° C. for about 24 hours to about 72 hours. If necessary, the culture can be aerated or agitated.

Where insect cells or insects are used as the host, the transformant is cultivated in, for example, Grace's Insect Medium (Grace, T. C. C., Nature, 195, 788 (1962)) to which an appropriate additive such as immobilized 10% bovine serum is added. Preferably, pH of the medium is adjusted to about 6.2 to about 6.4. Normally, the transformant is cultivated at about 27° C. for about 3 days to about 5 days and, if necessary, the culture may be aerated or agitated.

Where animal cells are employed as the host, the transformant is cultivated in, for example, MEM medium containing about 5% to about 20% fetal bovine serum [Science, 122, 501 (1952)], DMEM medium [Virology, 8, 396 (1959)], RPMI 1640 medium [The Journal of the American Medical Association, 199, 519 (1967)], 199 medium [Proceeding of the Society for the Biological Medicine, 73, 1 (1950)], etc. Preferably, pH of the medium is adjusted to about 6 to about 8. The transformant is usually cultivated at about 30° C. to about 40° C. for about 15 hours to about 60 hours and, if necessary, the culture may be aerated or agitated.

As described above, the receptor protein of the present invention can be produced in the cell or cell membrane, or outside the cell, of the transformant.

The receptor protein of the present invention can be separated and purified from the culture described above, e.g., by the following procedures.

When the receptor protein of the present invention is extracted from the culture or cells, after cultivation, the transformants or cells are collected by a publicly known method and suspended in a appropriate buffer. The transformants or cells are then disrupted by publicly known methods such as ultrasonication, a treatment with lysozyme and/or freeze-thaw cycling, followed by centrifugation, filtration, etc. Thus, the crude extract of the receptor protein can be obtained. The buffer used for the procedures may contain a protein modifier such as urea or guanidine hydrochloride, or a surfactant such as Triton X-100™, etc. When the receptor protein of the present invention is secreted in the culture broth, after completion of the cultivation, the supernatant can be separated from the transformants or cells to collect the supernatant by a publicly known method.

The supernatant or the receptor protein of the present invention contained in the extract thus obtained can be purified by appropriately combining the publicly known methods for separation and purification. Such publicly known methods for separation and purification include a method utilizing difference in solubility such as salting out, solvent precipitation, etc.; a method mainly utilizing difference in molecular weight such as dialysis, ultrafiltration, gel filtration, SDS-polyacrylamide gel electrophoresis, etc.; a method utilizing difference in electric charge such as ion exchange chromatography, etc.; a method utilizing difference in specific affinity such as affinity chromatography, etc.; a method utilizing difference in hydrophobicity such as reverse phase high performance liquid chromatography, etc.; a method utilizing difference in isoelectric point such as isoelectrofocusing electrophoresis; and the like.

When the receptor protein thus obtained is in a free form, it can be converted into the salt by publicly known methods or modifications thereof. On the other hand, when the receptor protein is obtained in the form of a salt, it can be converted into the free form or in the form of a different salt by publicly known methods or modifications thereof.

The receptor protein produced by the recombinant can be treated, prior to or after the purification, with an appropriate protein modifying enzyme so that the receptor protein can be appropriately modified to partially remove a polypeptide. Examples of the protein-modifying enzyme include trypsin, chymotrypsin, arginyl endopeptidase, protein kinase, glycosidase and the like.

The activity of the thus produced receptor protein of the present invention or salts thereof can be determined by a binding test to a labeled ligand and by an enzyme immunoassay using a specific antibody.

Antibodies to the receptor protein of the present invention, its partial peptide, or salts thereof may be any of polyclonal and monoclonal antibodies, so long as they can recognize the receptor protein of the present invention, its partial peptide, or salts thereof.

The antibodies to the receptor protein of the present invention, its partial peptide, or salts thereof (hereinafter sometimes referred to as the receptor protein of the present invention) can be manufactured according to publicly known methods for producing antibodies or antisera, using the receptor proteins of the present invention as antigens.

[Preparation of Monoclonal Antibody]

(a) Preparation of Monoclonal Antibody-Producing Cells

The receptor protein of the present invention is administered to warm-blooded animals either solely or together with carriers or diluents to the site where the production of antibody is possible by the administration. In order to potentiate the antibody productivity upon the administration, complete Freund's adjuvants or incomplete Freund's adjuvants may be administered. The administration is usually carried out once every two to six weeks and two to ten times in total. Examples of the applicable warm-blooded animals are monkeys, rabbits, dogs, guinea pigs, mice, rats, sheep and goats, with the use of mice and rats being preferred.

In the preparation of monoclonal antibody-producing cells, a warm-blooded animal, e.g., mice, immunized with an antigen wherein the antibody titer is noted is selected, then spleen or lymph node is collected after two to five days from the final immunization and antibody-producing cells contained therein are fused with myeloma cells from homozoic or heterozoic animal to give monoclonal antibody-producing hybridomas. Measurement of the antibody titer in antisera may be carried out, for example, by reacting a labeled receptor protein, which will be described later, with the antiserum followed by assaying the binding activity of the labeling agent bound to the antibody. The fusion may be carried out, for example, by the known method by Koehler and Milstein [Nature, 256, 495, (1975)]. Examples of the fusion promoting agent are polyethylene glycol (PEG), Sendai virus, etc., of which PEG is preferably employed.

Examples of the myeloma cells are those collected from warm-blooded animals such as NS-1, P3U1, SP2/0, AP-1, etc. In particular, P3U1 is preferably employed. A preferred ratio of the count of the antibody-producing cells used (spleen cells) to the count of myeloma cells is within a range of approximately 1:1 to 20:1. When PEG (preferably, PEG 1000 to PEG 6000) is added in a concentration of approximately 10 to 80% followed by culturing at approximately 20 to 40° C., preferably at approximately 30 to 37° C. for approximately 1 to 10 minutes, an efficient cell fusion can be carried out.

Various methods can be used for screening of a monoclonal antibody-producing hybridoma. Examples of such methods include a method which comprises adding the supernatant of hybridoma to a solid phase (e.g., a microplate) adsorbed with the receptor protein as an antigen directly or together with a carrier, adding an anti-immunoglobulin antibody (where mouse cells are used for the cell fusion, anti-mouse immunoglobulin antibody is used) labeled with a radioactive substance or an enzyme or Protein A and detecting the monoclonal antibody bound to the solid phase, a method which comprises adding the supernatant of hybridoma to a solid phase adsorbed with an anti-immunoglobulin antibody or protein A, adding the receptor protein labeled with a radioactive substance or an enzyme and detecting the monoclonal antibody bound to the solid phase, or the like.

The monoclonal antibody can be selected according to publicly known methods or their modifications. In general, the selection can be effected in a medium for animal cells supplemented with HAT (hypoxanthine, aminopterin and thymidine). Any selection and growth medium can be employed as far as the hybridoma can grow there. For example, RPMI 1640 medium containing 1% to 20%, preferably 10% to 20% fetal bovine serum, GIT medium (Wako Pure Chemical Industries, Ltd.) containing 1% to 10% fetal bovine serum, a serum free medium for cultivation of a hybridoma (SFM-101, Nissui Seiyaku Co., Ltd.) and the like can be used for the selection and growth medium. The cultivation is carried out generally at 20° C. to 40° C., preferably at about 37° C., for about 5 days to about 3 weeks, preferably 1 to 2 weeks, normally in 5% CO₂. The antibody titer of the culture supernatant of a hybridoma can be determined as in the assay for the antibody titer in antisera described above.

(b) Purification of Monoclonal Antibody

Separation and purification of a monoclonal antibody can be carried out as in the separation and purification of polyclonal antibodies, following the procedures for separation and purification of immunoglobulins [for example, salting-out, alcohol precipitation, isoelectric point precipitation, electrophoresis, adsorption and desorption with ion exchangers (e.g., DEAE), ultracentrifugation, gel filtration, or a specific purification method which comprises collecting only an antibody with an activated adsorbent such as an antigen-binding solid phase, protein A or protein G and dissociating the binding to obtain the antibody].

[Preparation of Polyclonal Antibody]

The polyclonal antibody of the present invention can be manufactured by publicly known methods or modifications thereof. For example, a complex of immunogen (antigen of the receptor protein) and a carrier protein is formed and a mammal is immunized with the complex in a manner similar to the method described above for the manufacture of monoclonal antibody. The product containing the antibody to the receptor protein of the present invention is collected from the immunized animal followed by separation and purification of the antibody.

In the complex of immunogen and carrier protein for immunizing mammal, the type of carrier protein and the mixing ratio of carrier to hapten may be any type and in any ratio, as long as the antibody is efficiently produced to the hapten immunized by crosslinking to the carrier. For example, bovine serum albumin, bovine thyroglobulin, keyhole limpet hemocyanin, etc. is coupled to hapten in a carrier-to-hapten weight ratio of approximately 0.1 to 20, preferably approximately 1 to 5.

A variety of condensation agents can be used for the coupling of carrier to hapten. Glutaraldehyde, carbodiimide, maleimide activated ester and activated ester reagents containing a thiol group, a dithiopyridyl group, etc. are used for the coupling. The condensation product is administered to warm-blooded animals either solely or together with carriers or diluents to the site that can produce the antibody by the administration. In order to potentiate the antibody productivity upon the administration, complete Freund's adjuvant or incomplete Freund's adjuvant may be administered. The administration is usually made once every about 2 to about 6 weeks and about 3 to about 10 times in total.

The polyclonal antibody can be collected from the blood, ascites, etc. of warm-blooded animal immunized by the method described above, preferably from the blood.

The polyclonal antibody titer in antiserum can be assayed by the same procedure as that for the determination of serum antibody titer described above. The separation and purification of the polyclonal antibody can be carried out, following the method for the separation and purification of immunoglobulins performed as in the separation and purification of monoclonal antibodies described hereinabove.

The receptor protein of the present invention or salts thereof, its partial peptide or salts thereof as well as the DNA encoding the receptor protein or its partial peptide can be used: (1) for determination of a ligand (agonist) to the receptor protein of the present invention, (2) as an agent for the prevention and/or treatment of disease associated with dysfunction of the receptor protein of the present invention, (3) as a genetic diagnostic agent, (4) for screening of a compound that alters the expression level of the receptor protein or its partial peptide of the present invention, (5) as an agent for the prevention and/or treatment of various diseases, comprising a compound that alters the expression level of the receptor protein or its partial peptide of the present invention, (6) for quantification of a ligand to the receptor protein of the present invention, (7) for screening of a compound (an agonist, an antagonist, etc.) that alters the binding property of the receptor protein of the present invention to a ligand, (8) as an agent for the prevention and/or treatment of various diseases, comprising a compound (an agonist, an antagonist, etc.) that alters the binding property of the receptor protein of the present invention to a ligand, (9) for quantification of the receptor protein or its partial peptide of the present invention, or salts thereof, (10) for screening a compound that alters the amount of the receptor protein or its partial peptide of the present invention in a cell membrane, (11) an agent for the prevention and/or treatment of various diseases comprising a compound that alters the amount of the receptor protein or its partial peptide of the present invention in a cell membrane, (12) for neutralization by antibodies to the receptor protein or its partial peptide or salts thereof according to the present invention, (13) for preparation of non-human animal bearing the DNA encoding the receptor protein of the present invention.

In particular, a compound (e.g., an agonist, an antagonist, etc.) that alters the binding property of a ligand to the G protein-coupled receptor protein of the present invention specific to human or other mammals can be screened by applying the receptor binding assay system using the expression system of the recombinant receptor protein of the present invention. The agonist or antagonist can be used as a prophylactic/therapeutic agent for various diseases.

Hereinafter, the receptor protein of the present invention or its partial peptide, or salts thereof (hereinafter sometimes merely referred to as the receptor protein of the present invention), the DNA encoding the receptor protein or its partial peptide of the present invention (hereinafter sometimes merely referred to as the DNA of the present invention) and the antibody to the receptor protein or its partial peptide of the present invention (hereinafter sometimes referred to as the antibody of the present invention) are specifically described in terms of their applications.

(1) Determination of a Ligand (Agonist) to the Receptor Protein of the Present Invention

The receptor protein of the present invention or its salts or the partial peptide of the present invention or its salts are useful as reagents for searching and determining a ligand (agonist) to the receptor protein of the present invention or salts thereof.

That is, the present invention provides a method for determining a ligand to the receptor protein of the present invention, which comprises contacting the receptor protein of the present invention or salts thereof or the partial peptide of the present invention or salts thereof with a test compound.

As the test compounds, there are used publicly known ligands (e.g., angiotensin, bombesin, canavinoid, cholecystokinin, glutamine, serotonin, melatonin, neuropeptide Y, opioid, purines, vasopressin, oxytocin, PACAP, secretin, glucagon, calcitonin, adrenomedulin, somatostatin, GHRH, CRF, ACTH, GRP, PTH, VIP (vasoactive intestinal and related polypeptide), somatostatin, dopamine, motilin, amylin, bradykinin, CGRP (calcitonin gene-related peptide), leukotrienes, pancreastatin, prostaglandins, thromboxane, adenosine, adrenaline, α and β-chemokines (e.g., IL-8, GROα, GROβ, GROγ, NAP-2, ENA-78, PF4, IP10, GCP-2, MCP-1, HC14, MCP-3, I-309, MIP-1α, MIP-1β, RANTES, etc.), endothelin, enterogastrin, histamine, neurotensin, TRH, pancreatic polypeptide, galanin, etc.) as well as other substances, for example, tissue extracts, cell culture supernatants, etc. from human or mammal (e.g., mice, rats, swine, bovine, sheep, monkeys, etc.). For example, the tissue extracts, cell culture supernatants, etc. are added to the receptor protein of the present invention and fractionated while assaying the cell-stimulating activity, etc. to finally give a single ligand.

Specifically, the method for determining ligands of the present invention comprises determining compounds (e.g., peptides, proteins, non-peptide compounds, synthetic compounds, fermentation products, etc.) or salts thereof that bind to the receptor protein of the present invention to provide cell stimulating activities (e.g., the activities that promote or suppress arachidonic acid release, acetylcholine release, intracellular Ca²⁺ release, intracellular cAMP production, intracellular cGMP production, inositol phosphate production, change in cell membrane potential, phosphorylation of intracellular proteins, activation of c-fos, pH reduction, etc.), either using the receptor, its partial peptide or salts thereof of the present invention, or using the constructed recombinant receptor protein expression system in the receptor binding assay system.

The method for determining the ligand of the present invention is characterized, for example, by measurement of the amount of a test compound bound to the receptor protein or its partial peptide of the present invention, the cell-stimulating activity, etc., when the receptor protein or its partial peptide of the present invention is brought in contact with the test compound.

More specifically, the present invention provides the following:

(1) A method for determining a ligand to the receptor protein of the present invention or salts thereof, which comprises measuring the amount of a labeled test compound bound to the protein or its salts of the present invention or the partial peptide or its salts of the present invention, when a labeled test compound is brought in contact with the receptor protein or its salts or the partial peptide or its salts;

(2) A method for determining a ligand to the receptor protein of the present invention or salts thereof, which comprises measuring the amount of a labeled test compound bound to a cell containing the receptor protein of the present invention or with a membrane fraction of the cell, when the labeled test compound is brought in contact with the cell or the membrane fraction;

(3) A method for determining a ligand to the receptor protein of the present invention, which comprises measuring the amount of a labeled test compound bound to the receptor protein or its salts, when the labeled test compound is brought in contact with the receptor protein expressed on the cell membrane by culturing a transformant containing a DNA encoding the receptor protein of the present invention;

(4) A method for determining a ligand to the receptor protein or its salts of the present invention, which comprises measuring the receptor protein-mediated cell stimulating activities (e.g., the activities that promote or suppress arachidonic acid release, acetylcholine release, intracellular Ca²⁺ release, intracellular cAMP production, intracellular cGMP production, inositol phosphate production, change in cell membrane potential, phosphorylation of intracellular proteins, activation of c-fos, pH reduction, etc.), when a test compound is brought in contact with the cell containing the receptor protein of the present invention; and,

(5) A method for determining a ligand to the receptor protein or its salts of the present invention, which comprises measuring the receptor protein-mediated cell stimulating activities (e.g., the activities that promote or suppress arachidonic acid release, acetylcholine release, intracellular Ca²⁺ release, intracellular cAMP production, intracellular cGMP production, inositol phosphate production, change in cell membrane potential, phosphorylation of intracellular proteins, activation of c-fos, pH reduction, etc.), when a test compound is brought in contact with the receptor protein expressed on a cell membrane by culturing a transformant containing a DNA encoding the receptor protein of the present invention.

In particular, it is preferred to perform the above-described methods (1) to (3) thereby to confirm that a test compound can bind to the receptor protein of the present invention, followed by the methods (4) and (5) described above.

A receptor protein used in ligand determining method may include any of ligands in which comprises a receptor protein or a partial peptide of the present invention. In addition, It prefers a receptor protein that largely expressed using animal cells.

The receptor protein of the present invention can be manufactured by the method for expression described above, preferably by expressing a DNA encoding the receptor protein in mammalian or insect cells. DNA fragments encoding the desired portion of the protein used include, but are not limited to, complementary DNAs. For example, gene fragments or synthetic DNAs may also be used. For introducing a DNA fragment encoding the receptor protein of the present invention into a host animal cell and efficiently expressing the same, it is preferred to insert the DNA fragment downstream the polyhedrin promoter of nuclear polyhedrosis virus (NPV), which is a baculovirus having insect hosts, an SV40-derived promoter, a retrovirus promoter, a metallothionein promoter, a human heat shock promoter, a cytomegalovirus promoter, an SRα promoter, or the like. The amount and quality of the receptor expressed can be determined by a publicly known method. For example, this determination can be made by the method described in the literature [Nambi, P. et al., J. Biol. Chem., Vol. 267, pp. 19555-19559 (1992)].

Accordingly, the subject containing the receptor protein in the method for determining the ligand may be the receptor protein purified by a publicly known method, cells containing the receptor protein or membrane fractions of such cells.

Where cells containing the receptor protein of the present invention are used in the method of the present invention for determination of ligands, the cells may be fixed using glutaraldehyde, formalin, etc. The fixation can be made by a publicly known method.

The cells containing the receptor protein of the present invention are host cells that have expressed the receptor protein of the present invention, which host cells include Escherichia coli, Bacillus subtilis, yeast, insect cells, animal cells, and the like.

The cell membrane fraction is a fraction abundant in cell membrane obtained by cell disruption and subsequent fractionation by a publicly known method. Useful cell disruption methods include cell squashing using a Potter-Elvehjem homogenizer, disruption using a Waring blender or Polytron (manufactured by Kinematica Inc.), disruption by ultrasonication, and disruption by cell spraying through thin nozzles under an increased pressure using a French press, or the like. Cell membrane fractionation is effected mainly by fractionation using a centrifugal force, such as centrifugation for fractionation and density gradient centrifugation. For example, cell disruption fluid is centrifuged at a low speed (500 rpm to 3,000 rpm) for a short period of time (normally about 1 to about 10 minutes), the resulting supernatant is then centrifuged at a higher speed (15,000 rpm to 30,000 rpm) normally for 30 minutes to 2 hours. The precipitate thus obtained is used as the membrane fraction. The membrane fraction is rich in the receptor protein expressed and membrane components such as cell-derived phospholipids, membrane proteins, etc.

The amount of the receptor protein in the cells containing the receptor protein and in the membrane fraction is preferably 10³ to 10⁸ molecules per cell, more preferably 10⁵ to 10⁷ molecules per cell. As the amount of expression increases, the ligand binding activity per unit of membrane fraction (specific activity) increases so that not only the highly sensitive screening system can be constructed but also large quantities of samples can be assayed with the same lot.

To perform the methods (1) through (3) for determination of a ligand to the receptor protein or its salts of the present invention, an appropriate receptor fraction and a labeled test compound are required.

The receptor protein fraction is preferably a fraction of naturally occurring receptor protein or a recombinant receptor fraction having an activity equivalent to thatlof the natural protein. Herein, the term “equivalent activity” is intended to mean a ligand binding activity, a signal transduction activity or the like that is equivalent to that possessed by naturally occurring receptor proteins.

Preferred examples of labeled test compounds include angiotensin, bombesin, canavinoid, cholecystokinin, glutamine, serotonin, melatonin, neuropeptide Y, opioid, purines, vasopressin, oxytocin, PACAP, secretin, glucagon, calcitonin, adrenomedulin, somatostatin, GHRH, CRF, ACTH, GRP, PTH, VIP (vasoactive intestinal and related polypeptide), somatostatin, dopamine, motilin, amylin, bradykinin, CGRP (calcitonin gene-related peptide), leukotrienes, pancreastatin, prostaglandins, thromboxane, adenosine, adrenaline, Ca and β-chemokines (e.g., IL-8, GROα, GROβ, GROγ, NAP-2, ENA-78, PF4, IP10, GCP-2, MCP-1, HC14, MCP-3, I-309, MIP-1α, MIP-1β, RANTES, etc.), endothelin, enterogastrin, histamine, neurotensin, TRH, pancreatic receptor protein, galanin, etc., which are labeled with [³H],[¹²⁵I],[¹⁴C],[³⁵S], etc.

Specifically, the ligand to the receptor protein or its salts of the present invention is determined by the following procedures. First, a standard receptor preparation is prepared by suspending cells containing the receptor protein of the present invention or the membrane fraction of the cells in a buffer appropriate for use in the determination method. Any buffer is usable so long as it does not interfere with ligand-receptor protein binding, such buffers including a phosphate buffer, a Tris-HCl buffer, etc., having a pH of 4 to 10 (preferably the pH of 6 to 8). For the purpose of minimizing non-specific binding, a surfactant such as CHAPS, Tween-80™ (manufactured by Kao-Atlas Inc.), digitonin, deoxycholate, etc. and various proteins such as bovine serum albumin, gelatin, etc. may optionally be added to buffers. Further for the purpose of suppressing the degradation of the receptor or ligand by a protease, a protease inhibitor such as PMSF, leupeptin, E-64 (manufactured by Peptide Institute, Inc.), pepstatin, etc. may also be added. A given amount (5,000 cpm to 500,000 cpm) of a test compound labeled with [³H], [¹²⁵I], [¹⁴C], [35S] or the like is added to 0.01 ml to 10 ml of the receptor solution. To determine the amount of non-specific binding (NSB), a reaction tube containing an unlabeled test compound in a large excess is also provided. The reaction is carried out at approximately 0° C. to 50° C., preferably about 4° C. to 37° C. for about 20 minutes to about 24 hours, preferably about 30 minutes to 3 hours. After completion of the reaction, the reaction mixture is filtrated through glass fiber filter paper, etc. and washed with an appropriate volume of the same buffer. The residual radioactivity in the glass fiber filter paper is then measured by means of a liquid scintillation counter or γ-counter. A test compound exceeding 0 cpm in count obtained by subtracting nonspecific binding (NSB) from the total binding (B) (B minus NSB) may be selected as a ligand (agonist) to the receptor protein or its salts of the present invention.

The above-described method (4) or (5) for determination of a ligand to the receptor protein or its salts of the present invention can be performed as follows. The receptor protein-mediated cell-stimulating activities (e.g., the activities that promote or suppress arachidonic acid release, acetylcholine release, intracellular Ca²⁺ release, intracellular cAMP production, intracellular cGMP production, inositol phosphate production, change in cell membrane potential, phosphorylation of intracellular proteins, activation of c-fos, pH reduction, etc.) may be determined by a publicly known method, or using an assay kit commercially available. Specifically, cells containing the receptor protein are first cultured on a multi-well plate, etc. Prior to the ligand determination, the medium is replaced with a fresh medium or with an appropriate non-cytotoxic buffer, followed by culturing for a given period of time in the presence of a test compound, etc. Subsequently, the cells are extracted or the supernatant is recovered and the resulting product is quantified by appropriate procedures. Where it is difficult to detect the production of the index substance for the cell-stimulating activity (e.g., arachidonic acid) due to a degrading enzyme contained in the cells, an inhibitor against such a degrading enzyme may be added prior to the assay. For detecting activities such as the cAMP production suppression activity, the baseline production in the cells is increased by forskolin or the like and the suppressing effect on the increased baseline production can then be detected.

The kit of the present invention for determination of the ligand that binds to the receptor protein or its salts of the present invention comprises the receptor protein or its salts of the present invention, the partial peptide or its salts of the present invention, the cell containing the receptor protein of the present invention, the membrane fraction of the cell containing the receptor protein of the present invention, or the like.

Examples of the ligand determination kit of the present invention are given below.

1. Reagents for Determining Ligands

(1) Buffers for Assay and Washing

Hanks' Balanced Salt Solution (manufactured by Gibco Co.) supplemented with 0.05% bovine serum albumin (Sigma Co.).

The solution is sterilized by filtration through a 0.45 μm filter and stored at 4° C. Alternatively, the solution may be prepared at use.

(2) Standard Receptor Protein Preparation

CHO cells on which the receptor protein of the present invention has been expressed are subjected to passage culture in a 12-well plate in a density of 5×10⁵ cells/well followed by culturing at 37° C. under 5% CO₂ and 95% air for 2 days.

(3) Labeled Test Compounds

Compounds labeled with commercially available [³H], [125I], [¹⁴C], [35S], etc. or compounds labeled by appropriate methods.

An aqueous solution of the compound is stored at 4° C. or −20° C. The solution is diluted to 1 μM with an assay buffer at use. A sparingly water-soluble test compound is dissolved in dimethylformamide, DMSO, methanol, etc.

(4) Non-Labeled Compounds

A non-labeled form of the same compound as the labeled compound is prepared in a concentration 100 to 1,000-fold higher than that of the labeled compound.

2. Method for Assay

(1) CHO cells expressing the receptor protein of the present invention are cultured in a 12-well culture plate. After washing twice with 1 ml of an assay buffer, 490 μl of the assay buffer is added to each well.

(2) After 5 μl of the labeled test compound is added, the resulting mixture is cultured at room temperature for an hour. To determine the non-specific binding, 5 μl of the non-labeled compound is added to the system.

(3) The reaction mixture is removed and the wells are washed 3 times with 1 ml of wash buffer. The labeled test compound bound to the cells is dissolved in 0.2N NaOH-1% SDS and then mixed with 4 ml of liquid scintillator A (manufactured by Wako Pure Chemical Industries, Ltd.).

(4) The radioactivity is measured using a liquid scintillation counter (manufactured by Beckman Co.).

The ligands that can bind to the receptor protein or its salts of the present invention include substances specifically present in the brain, pituitary gland and pancreas. Examples of such ligands are angiotensin, bombesin, canavinoid, cholecystokinin, glutamine, serotonin, melatonin, neuropeptide Y, opioids, purines, vasopressin, oxytocin, PACAP, secretin, glucagon, calcitonin, adrenomedulin, Dsomatostatin, GHRH, CRF, ACTH, GRP, PTH, VIP (vasoactive intestinal and related polypeptide), somatostatin, dopamine, motilin, amylin, bradykinin, CGRP (calcitonin gene-related peptide), leukotriens, pancreastatin, prostaglandins, thromboxane, adenosine, adrenaline, a and β-chemokines (e.g., IL-8, GROα, GROβ, GROγ, NAP-2, ENA-78, PF4, IP10, GCP-2, MCP-1, HC14, MCP-3, I-309, MIP-1α, MIP-1β, RANTES, etc.), endothelin, enterogastrin, histamine, neurotensin, TRH, pancreatic receptor protein, galanin, etc.

(2) Preventive and/or Therapeutic Agents for Diseases Associated with Dysfunction of the Receptor Protein of the Present Invention

In the method (1) described above, when the ligand to the receptor protein of the present invention is determined, the DNA encoding 1) the receptor protein of the present invention or 2) the receptor protein can be used as drugs such as preventive and/or therapeutic agents for diseases associated with dysfunction of the receptor protein of the present invention.

For example, when any physiological activity of the ligand cannot be expected in the body due to a reduced level of the receptor protein of the present invention in a patient (deficiency of the receptor protein), the ligand activity can be exhibited: (1) by administering the receptor protein of the present invention to the patient thereby to supplement the amount of the receptor protein; or (2) by increasing the amount of the receptor protein of the present invention in the patient (a) through administration of the DNA encoding the receptor protein of the present invention to the patient to effect expression, or b) through insertion of the DNA encoding the receptor protein of the present invention in the target cell to effect expression and then transplanting the cell thus expressed to the patient. Thus, the ligand activity can be exhibited to a sufficient extent. That is, the DNA encoding the receptor protein of the present invention is useful as a safe and low toxic preventive and/or therapeutic drug for diseases associated with the dysfunction of the receptor protein of the present invention.

The receptor protein of the present invention and the DNA encoding the receptor protein of the present invention is useful for the prevention and/or treatment of, e.g., hypertension, autoimmune disease, heart failure, cataract, glaucoma, acute bacterial meningitis, acute myocardial infarction, acute pancreatitis, acute viral encephalitis, adult respiratory distress syndrome, alcoholic hepatitis, Alzheimer's disease, asthma, arteriosclerosis, atopic dermatitis, bacterial pneumonia, bladder cancer, fracture, breast cancer, bulimia, polyphagia, burn healing, uterine cervical cancer, chronic lymphocytic leukemia, chronic myelogenous leukemia, chronic pancreatitis, liver cirrhosis, cancer of the colon and rectum (colon cancer/rectal cancer), Crohn's disease, dementia, diabetic complications, diabetic nephropathy, diabetic neuropathy, diabetic retinopathy, gastritis, Helicobacter pylori bacterial infectious disease, hepatic insufficiency, hepatitis A, hepatitis B, hepatitis C, hepatitis, herpes simplex virus infectious disease, varicellazoster virus infectious disease, Hodgkin's disease, AIDS infectious disease, human papilloma virus infectious disease, hypercalcemia, hypercholesterolemia, hyperglyceridemia, hyperlipemia, infectious disease, influenza infectious disease, insulin dependent diabetes mellitus (type I), invasive staphylococcal infectious disease, malignant melanoma, cancer metastasis, multiple myeloma, allergic rhinitis, nephritis, non-Hodgkin's lymphoma, insulin-independent diabetes mellitus (type II), non-small cell lung cancer, organ transplantation, arthrosteitis, osteomalacia, osteopenia, osteoporosis, ovarian cancer, Behcet's disease of bone, peptic ulcer, peripheral vessel disease, prostatic cancer, reflux esophagitis, renal insufficiency, rheumatoid arthritis, schizophrenia, sepsis, septic shock, severe systemic fungal infectious disease, small cell lung cancer, spinal injury, stomach cancer, systemic lupus erythematosus, transient cerebral ischemia, tuberculosis, cardiac valve failure, vascular/multiple infarction dementia, wound healing, insomnia, arthritis, pituitary hormone secretion disorder, pollakiuria, uremia, neurodegenerative disease, etc.

Where the receptor protein of the present invention is used as the preventive/therapeutic agent described above, the receptor protein can be prepared into a pharmaceutical preparation in a conventional manner.

On the other hand, when the DNA encoding the receptor protein of the present invention (hereinafter sometimes referred to as the DNA of the present invention) is used as the preventive/therapeutic agent described above, the DNA of the present invention may be used alone or after inserting it into a appropriate vector such as retrovirus vector, adenovirus vector or adenovirus-associated virus vector followed by a conventional means for drug administration. The DNA of the present invention may also be administered as intact DNA, or with adjuvants to assist its uptake by gene gun or through a catheter such as a catheter with a hydrogel.

For example, (1) the receptor protein of the present invention or (2) the DNA encoding the receptor protein can be used orally in the form of tablets which may be sugar coated if necessary and desired, capsules, elixirs, microcapsules etc., or parenterally in the form of injectable preparations such as a sterile solution, a suspension, etc. in water or with other pharmaceutically acceptable liquid. These preparations can be manufactured, e.g., by mixing (1) the receptor protein of the present invention or (2) the DNA encoding the receptor protein, with a physiologically acceptable known carrier, flavoring agent, excipient, vehicle, antiseptic, stabilizer, binder, etc. in a unit dosage form required in a generally accepted manner applied to making pharmaceutical preparations. The active ingredient in the preparation is controlled in such a dose that an appropriate dose is obtained within the specified range given.

Additives miscible with tablets, capsules etc. include a binder such as gelatin, corn starch, tragacanth and gum arabic, an excipient such as crystalline cellulose, a swelling agent such as corn starch, gelatin and alginic acid, a lubricant such as magnesium stearate, a sweetening agent such as sucrose, lactose and saccharin, and a flavoring agent such as peppermint, akamono oil and cherry. When the unit dosage is in the form of capsules, liquid carriers such as oils and fats may further be used together with the additives described above. A sterile composition for injection may be formulated according to a conventional manner used to make pharmaceutical compositions, e.g., by dissolving or suspending the active ingredients in a vehicle such as water for injection with a naturally occurring vegetable oil such as sesame oil and coconut oil, etc. to prepare the pharmaceutical composition. Examples of an aqueous medium for injection include physiological saline and an isotonic solution containing glucose and other auxiliary agents (e.g., D-sorbitol, D-mannitol and sodium chloride) and may be used in combination with an appropriate dissolution aid such as an alcohol (e.g., ethanol), a polyalcohol (e.g., propylene glycol and polyethylene glycol), a nonionic surfactant (e.g., polysorbate 80™ and HCO-50), etc. As an oily medium, for example, sesame oil and soybean oil may be used, which can be used in combination with a dissolution aid such as benzyl benzoate, benzyl alcohol, etc.

The prophylactic/therapeutic agent described above may also be formulated with a buffer (e.g., phosphate buffer and sodium acetate buffer) a soothing agent (e.g., benzalkonium chloride, procaine hydrochloride, etc.), a stabilizer (e.g., human serum albumin, polyethylene glycol, etc.), a preservative (e.g., benzyl alcohol, phenol, etc.), an antioxidant, and the like. The thus-prepared liquid injection is normally filled in an appropriate ampoule.

Since the pharmaceutical preparation thus obtained is safe and low toxic, the preparation can be administered, for example, to human or mammals (e.g., rat, rabbit, sheep, swine, bovine, cat, dog, monkey, etc.).

The dose of the receptor protein of the present invention varies depending on subject to be administered, target organ, symptom, method for administration, etc.; in oral administration, the dose is normally about 0.1 mg to about 100 mg, preferably about 1.0 mg to about 50 mg, and more preferably about 1.0 mg to about 20 mg per day for a patient with schizophrenia (weighing 60 kg). In parenteral administration, the single dose varies depending on subject to be administered, target organ, symptom, route for administration, etc. but it is advantageous to administer the receptor protein intravenously at a daily dose of about 0.01 mg to about 30 mg, preferably about 0.1 mg to about 20 mg, and more preferably about 0.1 mg to about 10 mg for a patient with hypertension (weighing 60 kg). For other animal species, the corresponding dose as converted per 60 kg weight can be administered.

The dose of the DNA of the present invention varies depending on subject to be administered, target organ, symptom, route for administration, etc.; in oral administration, the dose is normally about 0.1 mg to about 100 mg, preferably about 1.0 to about 50 mg, and more preferably about 1.0 to about 20 mg per day for a patient with hypertension (weighing 60 kg). In parenteral administration, the single dose varies depending on subject to be administered, target organ, symptom, route for administration, etc. but it is advantageous to administer the DNA intravenously at a daily dose of about 0.01 to about 30 mg, preferably about 0.1 to about 20 mg, and more preferably about 0.1 to about 10 mg for a patient with cancer (weighing 60 kg). For other animal species, the corresponding dose as converted per 60 kg weight can be administered.

(3) Gene Diagnostic Agent

The DNA of the present invention can detect an abnormality (gene abnormality) of the DNA or mRNA encoding the receptor protein or its partial peptide of the present invention in human or mammal (e.g., rat, rabbit, sheep, swine, bovine, cat, dog, monkey, etc.) by using the same as a probe. Therefore, the DNA is useful as a gene diagnostic agent for damages to the DNA or mRNA, mutation thereof, or decreased expression thereof, or increased expression or overexpression of the DNA or mRNA.

The gene diagnosis described above using the DNA of the present invention can be performed by, for example, publicly known Northern hybridization assay or PCR-SSCP assay (Genomics, 5, 874-879 (1989); Proceedings of the National Academy of Sciences of the United States of America, 86, 2766-2770 (1989)), or the like.

(4) Method of Screening Compounds that Alter the Expression Level of the Receptor Protein or its Partial Peptide of the Present Invention

Using as a probe, the DNA of the present invention can be used for screening of compounds that alter the expression level of the receptor protein or its partial peptide of the present invention.

That is, the present invention provides method of screening compounds that alter the expression level of the receptor protein or its partial peptide of the present invention, by measuring the mRNA level of the receptor protein or its partial peptide of the present invention contained in, for example, (i) \mathring{1} blood, \mathring{2} specific organs, and \mathring{3} tissues or cells isolated from the organs of non-human mammals and (ii) transformants, etc.

Specifically, the mRNA level of the receptor protein or its partial peptide of the present invention is measured as follows.

(i) Normal or non-human animals of disease models (e.g., mice, rats, rabbits, sheep, swine, bovine, cats, dogs, monkeys, etc., more specifically, dementia rats, obese mice, arteriosclerosis rabbits, tumor-bearing mice, etc.) receive administration of a drug (e.g., neurotropic drugs, hypotensive drugs, anticancer drugs, antiobestic drugs, etc.) or physical stress (e.g., soaking stress, electric shock, light and darkness, low temperatures, etc.), and the blood, specific organs (e.g., brain, liver, kidneys, etc.), or tissues or cells isolated from the organs are collected after lapse of a specified time.

The mRNA of the receptor protein or its partial peptide of the present invention contained in the obtained cells is extracted, for example, in a conventional manner, quantified using, for example, TaqManPCR, etc., and may be analyzed by Northern blotting using publicly known methods.

(ii) Transformants capable of expressing the receptor protein or its partial peptide of the present invention are prepared following the methods described above, and the mRNA of the receptor protein or its partial peptide of the present invention contained in the transformants can be quantified and analyzed as described above.

The compounds that alter the expression level of the receptor protein or its partial peptide of the present invention can be screened by the following procedures.

(i) To normal or non-human mammals of disease models, a test compound is administered at a specified time before (30 minutes to 24 hours before, preferably 30 minutes to 12 hours before, more preferably 1 hour to 6 hours before), at a specified time after (30 minutes to 3 days after, preferably 1 hour to 2 days after, more preferably 1 hour to 24 hours after), or simultaneously with a drug, physical stress or the like. At a specified time (30 minute to 3 days, preferably 1 hour to 2 days, more preferably 1 hour to 24 hours) after administration of the test compound, the mRNA level of the receptor protein or its partial peptide of the present invention contained in cells are quantified and analyzed.

(ii) Transformants are cultured in a conventional manner and a test compound is mixed in a culture medium. After a specified time (1 day to 7 days after, preferably 1 day to 3 days after, more preferably 2 days or 3 days after), the mRNA level of the receptor protein or its partial peptide of the present invention contained in the transformant can be quantified and analyzed.

The compounds or salts thereof obtainable by the screening method of the present invention are compounds that alter the expression level of the receptor protein or its partial peptide of the present invention, and are specifically; (a) compounds that potentiate the receptor protein-mediated cell-stimulating activities (e.g., the activities that promote or suppress arachidonic acid release, acetylcholine release, intracellular Ca²⁺ release, intracellular cAMP production, intracellular cGMP production, inositol phosphate production, change in cell membrane potential, phosphorylation of intracellular proteins, activation of c-fos, pH reduction, etc.) by increasing the expression level of the receptor protein or its partial peptide of the present invention; and (b) compounds that decrease the cell-stimulating activities by reducing the expression level of the receptor protein or its partial peptide of the present invention.

The compounds include peptides, proteins, non-peptide compounds, synthetic compounds, fermentation products, and the like. These compounds may be novel or known compounds.

The compounds that potentiate the cell-stimulating activities are useful as safe and low-toxic pharmaceuticals for the physiological activities of the receptor protein of the present invention (for the prevention and/or treatment of, e.g., hypertension, autoimmune disease, heart failure, cataract, glaucoma, acute bacterial meningitis, acute myocardial infarction, acute pancreatitis, acute viral encephalitis, adult respiratory distress syndrome, alcoholic hepatitis, Alzheimer's disease, asthma, arteriosclerosis, atopic dermatitis, bacterial pneumonia, bladder cancer, fracture, breast cancer, bulimia, polyphagia, burn healing, uterine cervical cancer, chronic lymphocytic leukemia, chronic myelogenous leukemia, chronic pancreatitis, liver cirrhosis, cancer of the colon and rectum (colon cancer/rectal cancer), Crohn's disease, dementia, diabetic complications, diabetic nephropathy, diabetic neuropathy, diabetic retinopathy, gastritis, Helicobacter pylori bacterial infectious disease, hepatic insufficiency, hepatitis A, hepatitis B, hepatitis C, hepatitis, herpes simplex virus infectious disease, varicellazoster virus infectious disease, Hodgkin's disease, AIDS infectious disease, human papilloma virus infectious disease, hypercalcemia, hypercholesterolemia, hyperglyceridemia, hyperlipemia, infectious disease, influenza infectious disease, insulin dependent diabetes mellitus (type I), invasive staphylococcal infectious disease, malignant melanoma, cancer metastasis, multiple myeloma, allergic rhinitis, nephritis, non-Hodgkin's lymphoma, insulin-independent diabetes mellitus (type II), non-small cell lung cancer, organ transplantation, arthrosteitis, osteomalacia, osteopenia, osteoporosis, ovarian cancer, Behcet's disease of bone, peptic ulcer, peripheral vessel disease, prostatic cancer, reflux esophagitis, renal insufficiency, rheumatoid arthritis, schizophrenia, sepsis, septic shock, severe systemic fungal infectious disease, small cell lung cancer, spinal injury, stomach cancer, systemic lupus erythematosus, transient cerebral ischemia, tuberculosis, cardiac valve failure, vascular/multiple infarction dementia, wound healing, insomnia, arthritis, pituitary hormone secretion disorder, pollakiuria, uremia, neurodegenerative disease, etc.).

The compounds that decrease the cell-stimulating activities are useful as safe and low-toxic pharmaceuticals for reducing the physiological activities of the receptor protein of the present invention.

When the compound or salts thereof, which can be obtained by the screening method of the present invention, are used as pharmaceutical compositions, a conventional means may be applied to making pharmaceutical preparations. For example, the compound or its salts may be prepared into tablets, capsules, elixirs, microcapsules, sterile solutions, suspensions, etc., as in the pharmaceuticals containing the receptor protein of the present invention described above.

Since the thus obtained preparations are safe and low toxic, they may be administered to human or mammals (e.g., rat, rabbit, sheep, swine, bovine, cat, dog, monkey, etc.).

The dose of the compound or salts thereof varies depending on subject to be administered, target organ, symptom, method for administration, etc.; for example, in oral administration, the dose is normally about 0.1 to about 100 mg, preferably about 1.0 to about 50 mg, more preferably about 1.0 to about 20 mg per day for the patient with hypertension (as 60 kg body weight). In parenteral administration, the single dose varies depending on subject to be administered, target organ, symptom, method for administration, etc. but it is advantageous to administer the compound intravenously to the patient with hypertension (as 60 kg body weight) at a daily dose of about 0.01 to about 30 mg, preferably about 0.1 to about 20 mg, more preferably about 0.1 to about 10 mg. For other animal species, the corresponding dose as converted per 60 kg weight can be administered.

(5) Prophylactic and/or Therapeutic Drugs for Various Diseases, Containing Compounds that Alter the Expression Level of the Receptor Protein or its Partial Peptide of the Present Invention

The compounds that alter the expression level of the receptor protein or its partial peptide of the present invention can be used as prophylactic and/or therapeutic drugs for diseases associated with dysfunction of the receptor protein of the present invention.

When the compounds are used as prophylactic and/or therapeutic drugs for diseases associated with dysfunction of the receptor protein of the present invention, the compounds can be prepared into pharmaceutical preparations by a conventional means.

For example, the compounds can be administered orally in the form of a sugar-coated dragee, capsule, elixir, microcapsule, etc., or parenterally in the form of injectable preparations such as a sterile solution, a suspension, etc., in water, or together with other pharmaceutically acceptable liquid. For example, these preparations can be manufactured by mixing the compounds with physiologically acceptable known carrier, flavor, excipient, vehicle, antiseptic, stabilizer, binder, etc. in a unit dosage form required for generally approved drug preparations. The amount of the active ingredient in these preparations is set to an appropriate dose within the indicated range.

For the additives that may be mixed in tablets, capsules, etc., for example, binders such as gelatin, cornstarch, tragacanth or gum arabic, excipients such as crystalline cellulose, swelling agents such as corn starch, gelatin, alginic acid, etc., lubricants such as magnesium stearate, sweeteners such as sucrose, lactose, saccharin, etc., and flavors such as peppermint, akamono oil, cherry, etc. are used. When the dosage form is a capsule, a liquid carrier such as fat and oil may further be contained, in addition to the materials described above. Sterile compositions for injection can be formulated following the conventional preparation procedures such as dissolving or suspending the active substance in a vehicle, e.g., water for injection, naturally occurring vegetable oils, e.g., sesame oil, coconut oil, etc. As the aqueous solution for injection, for example, physiological saline, isotonic solutions (e.g., D-sorbitol, D-mannitol, sodium chloride, etc.) containing glucose and other aids are used. Appropriate dissolution-assisting agents, for example, an alcohol (e.g., ethanol), a polyalcohol (e.g., propylene glycol, polyethylene glycol), a nonionic surfactant (e.g., polysorbate 80™, HCO-50), etc. may be used in combination. For the oily solution, for example, sesame oil, soybean oil and the like are used, and dissolution-assisting agents such as benzyl benzoate, benzyl alcohol, etc. may be used in combination.

The prophylactic/therapeutic drugs described above may be formulated together with buffers (e.g., phosphate buffer, sodium acetate buffer), pain killers (e.g., benzalkonium chloride, procaine hydrochloride), stabilizers (e.g., human serum albumin, polyethylene glycol, etc.), preservatives (e.g., benzyl alcohol, phenol, etc.), antioxidants, and the like. The injection preparations prepared are usually filled in appropriate ampoules.

The pharmaceutical preparations thus obtained are safe and low-toxic, and can be administered to, for example, human and mammal (e.g., rats, rabbits, sheep, swine, bovine, cats, dogs, monkeys, etc.).

The dosage of the compounds or its salts differs depending on subject to be administered, target organ, symptom, administration method, etc. The dose of the compound or salts thereof varies depending on subject to be administered, target organ, symptom, method for administration, etc.; for example, in oral administration, the dose is generally about 0.1 to about 100 mg, preferably about 1.0 to about 50 mg, more preferably about 1.0 to about 20 mg per day for the patient with hypertension (as 60 kg body weight). In parenteral administration, the single dose varies depending on subject to be administered, target organ, symptom, method for administration, etc. but it is advantageous to administer the compound intravenously to the patient with hypertension (as 60 kg body weight) at a daily dose of about 0.01 to about 30 mg, preferably about 0.1 to about 20 mg, more preferably about 0.1 to about 10 mg. For other animal species, the corresponding dose as converted per 60 kg weight can be administered.

(6) Quantification of Ligands to the Receptor Protein of the Present Invention

Since the receptor protein of the present invention possesses the ligand-binding activity, the ligand concentration in the body can be quantified with good sensitivity.

The quantification method of the present invention can be used in combination with, for example, the competitive method. That is, the ligand concentration in a specimen can be measured by contacting the specimen to the receptor protein of the present invention. Specifically, the quantification can be made in accordance with, for example, the methods described in (1) and (2) below or its modified methods.

(1) Hiroshi Irie, ed., ‘Radioimmunoassay,’ (Kodansha, published 1974)

(2) Hiroshi Irie, ed., ‘Sequel to the Radioimmunoassay,’ (Kodansha, published 1979)

(7) Method of Screening the Compounds (Agonists, Antagonists, etc.) that Alter the Binding Property of the Receptor Protein of the Present Invention to Ligands

Using the receptor protein of the present invention, or using the receptor binding assay system consisting of the expression system constructed by a recombinant receptor protein, the compounds (e.g., peptides, proteins, non-peptide compounds, synthetic compounds, fermentation products, etc.) or salts thereof that alter the binding property of ligands to the receptor protein can be efficiently screened.

Such compounds include (a) compounds that have the G protein-coupled receptor-mediated cell-stimulating activities (e.g., the activities that promote or suppress arachidonic acid release, acetylcholine release, intracellular Ca²⁺ release, intracellular cAMP production, intracellular cGMP production, inositol phosphate production, alters in cell membrane potential, phosphorylation of intracellular proteins, activation of c-fos, pH reduction, etc.) (so-called agonists to the receptor protein of the present invention); (b) the compounds that do not have the cell-stimulating activities (so-called antagonists to the receptor protein of the present invention); (c) the compounds that increase the binding force between ligands and the receptor protein of the present invention; or (d) the compounds that decrease the binding force between ligands and the receptor protein of the present invention (it is preferred to screen the compounds described in (a) using the ligand determination methods described above).

That is, the present invention provides a method of screening the compound and its salts that alter the binding property between ligands and the receptor protein or its partial peptide or salts thereof, which comprises comparing (i) the case in which the receptor protein or its partial peptide or its salts are brought in contact with a ligand, and (ii) the case in which the receptor protein or its partial peptide or its salts are brought in contact with a test compound and the ligand.

The screening method of the present invention are characterized by measuring and comparing, for example, the amount of a ligand bound to the receptor protein or the cell-stimulating activity between the cases (i) and (ii).

More specifically, the present invention provides the following features.

(1) A method of screening a compound or its salts that alter the binding property between a ligand and the receptor protein of the present invention, which comprises measuring and comparing the amount of a labeled ligand bound to the receptor protein, wherein the labeled ligand is brought in contact with the receptor protein of the present invention, and the amount of a labeled ligand bound to the receptor protein of the present invention, wherein the labeled ligand and a test compound are brought in contact with the receptor protein of the present invention;

(2) A method of screening a compound or its salts that alter the binding property between a ligand and the receptor protein of the present invention, which comprises measuring and comparing the amount of a labeled ligand bound to a cell containing the receptor protein of the present invention or the membrane fraction of the cell, wherein the labeled ligand is brought in contact with the cell or its membrane fraction, and the amount of a labeled ligand bound to a cell containing the receptor protein of the present invention or the membrane fraction of the cell, wherein the labeled ligand and a test compound are brought in contact with the cell or its membrane fraction;

(3) A method of screening a compound or its salts that alter the binding property between a ligand and the receptor protein of the present invention, which comprises measuring and comparing the amount of a labeled ligand bound to the receptor protein, wherein the labeled ligand is brought in contact with the receptor protein of the present invention expressed on the cell membrane by culturing a transformant containing the DNA of the present invention, and the amount of the labeled ligand bound to the receptor protein, wherein the labeled ligand and a test compound are brought in contact with the receptor protein of the present invention expressed on the cell membrane by culturing a transformant containing the DNA of the present invention;

(4) A method of screening a compound or its salts that alter the binding property between a ligand and the receptor protein of the present invention, which comprises measuring and comparing the receptor-mediated cell stimulating activity (e.g., the activities that promote or suppress arachidonic acid release, acetylcholine release, intracellular Ca²⁺ release, intracellular CAMP production, intracellular cGMP production, inositol phosphate production, alters in cell membrane potential, phosphorylation of intracellular proteins, activation of c-fos, pH reduction, etc.) when a compound (e.g., a ligand to the receptor protein of the present invention, etc.) that activates the receptor protein of the present invention is brought in contact with a cell containing the receptor protein of the present invention, and the receptor-mediated cell stimulating activity when a compound that activates the receptor protein of the present invention and a test compound are brought in contact with a cell containing the receptor protein of the present invention; and,

(5) A method of screening a compound or its salts that alter the binding property between a ligand and the receptor protein of the present invention, which comprises measuring and comparing the receptor-mediated cell stimulating activity (e.g., the activities that promote or suppress arachidonic acid release, acetylcholine release, intracellular Ca²⁺ release, intracellular cAMP production, intracellular cGMP production, inositol phosphate production, alters in cell membrane potential, phosphorylation of intracellular proteins, activation of c-fos, pH reduction, etc.) when a compound (e.g., a ligand to the receptor protein of the present invention, etc.) that activates the receptor protein of the present invention is brought in contact with the receptor protein of the present invention expressed on the cell membrane by culturing a transformant containing the DNA of the present invention, and the receptor-mediated cell stimulating activity when a compound that activates the receptor protein of the present invention and a test compound are brought in contact with the receptor protein of the present invention expressed on the cell membrane by culturing a transformant containing the DNA of the present invention.

Before the receptor protein of the present invention were obtained and when G protein-coupled receptor agonists or antagonists were screened, candidate compounds were first obtained using cells or tissues containing the receptor protein from rats, etc. (primary screening). Then, candidate compounds needed to be examined if the compounds actually inhibit the binding between human G protein-coupled receptor protein and ligands (secondary screening). When cells, tissues or cell membrane fractions were directly used, other receptor proteins were also intermingled so that it was difficult to screen agonists or antagonists to the objective receptor protein.

However, using, e.g., the receptor protein of the present invention, the primary screening becomes unnecessary, and the compound that inhibits the binding of a ligand to the receptor protein can be efficiently screened. Furthermore, it is easy to evaluate whether the obtained compound is an agonist or an antagonist.

The screening method of the present invention is specifically described below.

First, for the receptor protein of the present invention used for the screening method of the present invention, any substance may be usable as far as it contains the receptor protein of the present invention described above, but preferred is the cell membrane fraction of mammalian organs containing the receptor protein of the present invention. However, since it is extremely difficult to obtain human organs, rat-derived receptor proteins expressed in a large scale using recombinants are preferably used for screening.

To manufacture the receptor protein of the present invention, the procedures described above are used, and it is preferred to express the DNA of the present invention in mammalian or insect cells. For the DNA fragment encoding the objective protein region, the complementary DNA is used, but the DNA fragment is not necessarily limited to the complementary DNA. For example, the gene fragments or synthetic DNA may be used. To introduce a DNA fragment encoding the receptor protein of the present invention into host animal cells and efficiently express the DNA, it is preferred to insert the DNA fragment at the downstream of polyhedorin promoter of nuclear polyhedrosis virus (NPV) belonging to baculovirus hosted by insects, SV40-derived promoter, retrovirus promoter, metallothionein promoter, human heat shock promoter, cytomegalovirus promoter, SRα promoter, etc. The amount and quality of the expressed receptor are examined by publicly known methods, for example, the method described in the literature [Nambi, P. et al., The Journal of Biological Chemistry (J. Biol. Chem.) Vol. 267; 19555-19559, 1992].

Therefore, in the screening method of invention, the material containing the receptor protein of the present invention may be the receptor protein purified by publicly known methods; alternatively, cells containing the receptor protein and the cell membrane fraction containing the receptor protein may also be used.

In the screening method of the present invention, when cells containing the receptor protein of the present invention are used, the cells may be fixed with glutaraldehyde, formalin, etc. Fixation can be effected in accordance with publicly known methods.

The cells containing the receptor protein of the present invention refer to host cells that have expressed the receptor protein. Preferred examples of the host cells are Escherichia coli, Bacillus subtilis, yeast, insect cells, animal cells, etc.

The cell membrane fraction refers to a fraction abundant in cell membranes obtained after destruction of the cells by publicly known methods. The cell destruction methods include cell squashing using a Potter-Elvehjem homogenizer, disruption using a Waring blender or Polytron (manufactured by Kinematica Inc.), disruption by ultrasonication, disruption by cell spraying through thin nozzles under an increased pressure using a French press, or the like. Fractionation of cell membranes is effected mainly by fractionation using a centrifugal force, such as centrifugation for fractionation and density gradient centrifugation. For example, cell disruption fluid is centrifuged at a low speed (500 rpm to 3,000 rpm) for a short period of time (normally about 1 to about 10 minutes), the resulting supernatant is then centrifuged at a higher speed (15,000 rpm to 30,000 rpm) normally for 30 minutes to 2 hours. The precipitate thus obtained is used as the membrane fraction. The membrane fraction is rich in the receptor protein expressed and membrane components such as cell-derived phospholipids, membrane proteins, etc.

The amount of the receptor protein in the cells containing the receptor protein and in the membrane fraction is preferably 10³ to 10⁸ molecules per cell, more preferably 10⁵ to 10⁷ molecules per cell. As the amount of expression increases, the ligand binding activity per unit of membrane fraction (specific activity) increases so that not only the highly sensitive screening system can be constructed but also large quantities of samples can be assayed with the same lot.

To screen compounds that alter the binding property between ligands and the receptor protein of the present invention described in (1) to (3) described above, for example, appropriate receptor protein fractions and labeled ligands are necessary.

For the receptor protein fraction, native receptor protein fractions or recombinant receptor protein fractions with an activity equivalent to that of the native receptor protein fraction are preferred. The equivalent activity means equivalent ligand-binding activity, signal transduction activity, or the like. For the labeled ligand, labeled ligands and labeled ligand analogues are used. For example, ligands labeled with [³H], [125I],[¹⁴C], [³⁵S], etc. are used.

Specifically, for screening of the compounds that alter the binding property between ligands and the receptor protein of the present invention, the receptor protein standard is first prepared by suspending cells containing the receptor protein of the present invention or a membrane fraction of the cells in a buffer appropriate for screening. Any buffer that does not inhibit the binding of ligands to the receptor protein, such as phosphate buffer, Tris-hydrochloride buffer, etc. having a pH of 4 to 10 (preferably a pH of 6 to 8), can be used. To minimize any non-specific binding, a surfactant such as CHAPS, Tween-80™ (Kao-Atlas Co.), digitonin, deoxycholate, etc. may be added to the buffer. For the purpose of preventing degradation of the receptor and ligands by proteases, protease inhibitors such as PMSF, leupeptin, E-64 (Peptide Research Laboratory, Co.), pepstatin, etc. may be added. To 0.01 ml to 10 ml of the receptor solution, a specified amount (5,000 cpm to 500,000 cpm) of a labeled ligand is added, and a test compound of 10⁻⁴ M to 10⁻¹⁰ M is allowed to be co-present therewith. To examine the non-specific binding (NSB), reaction tubes charged with a large excess of the non-labeled ligand are also prepared. The reaction is performed at about 0° C. to about 50° C., preferably about 4° C. to about 37° C., for about 20 minutes to about 24 hours, preferably for about 30 minutes to about 3 hours. After the reaction, the reaction solution is filtered through a glass fiber filter and the filter is washed with an appropriate volume of the buffer. The radioactivity remaining on the glass fiber filter is then measured using a liquid scintillation counter or a γ-counter. Taking as 100% the count obtained by subtracting the amount of non-specific binding (NSB) from the count obtained in the absence of any competitive substance (B₀), the test compound can be selected as a candidate substance having a potential of competitive inhibition, when the amount of specific binding (B-NSB) is, for example, 50% or less.

To perform the method of screening compounds that alter the binding property between ligands and the receptor protein of the present invention in (4) and (5) described above, for example, the receptor protein-mediated cell-stimulating activities (e.g., the activities that promote or suppress arachidonic acid release, acetylcholine release, intracellular Ca²⁺ release, intracellular cAMP production, intracellular cGMP production, inositol phosphate production, changes in cell membrane potential, phosphorylation of intracellular proteins, activation of c-fos, pH reduction, etc.) can be assayed using publicly known methods or assay kits commercially available.

Specifically, cells containing the receptor protein of the present invention are first cultured in a multiwell plate, etc. Before the screening, the medium is exchanged to a fresh medium or an appropriate buffer that is not toxic to the cells. After the cells are incubated together with a test compound for a given period of time, the cells are extracted or the supernatant is collected, and the product is quantified by the corresponding method. When the formation of an indicator substance for the cell-stimulating activities (e.g., arachidonic acid, etc.) is detected only with difficulty due to degrading enzymes contained in the cells, inhibitors of the degrading enzymes may be added to proceed the assay. As for the cAMP production inhibiting activity, the baseline production in the cells is increased by forskolin, etc., to detect the inhibiting activity as an effect on the cells with the increased baseline production.

The screening through assaying the cell-stimulating activities require cells that have expressed an appropriate receptor protein. As the cells that have expressed the receptor protein of the present invention, the cell line possessing the native type of the receptor protein of the present invention, the cell line that have expressed the recombinant receptor protein described above, and the like, are desirable.

Examples of the test compounds include peptides, proteins, non-peptide compounds, synthetic compounds, fermentation products, cell extracts, plant extracts, animal tissue extracts, and the like. These compounds may be novel or known compounds.

The kit for screening the compound or its salts that alter the binding property between ligands and the receptor protein of the present invention comprises the receptor protein of the present invention, the cell containing the receptor protein of the present invention, the membrane fraction of the cell containing the receptor protein of the present invention, or the like.

Examples of the screening kits of the present invention are as follow.

1. Reagents for Screening

(1) Buffers for Assaying and Washing

Hanks' balanced salt solution (Gibco Co.) supplemented with 0.05% bovine serum albumin (Sigma Co.).

The solution is sterilized by filtration through a 0.45 μm filter, and stored at 4° C. or may be prepared at use.

(2) Standard G Protein-Coupled Receptor

CHO cells that have expressed the receptor protein of the present invention are seeded in 12-well plates at a density of 5×10⁵ cells/well and cultured at 37° C. under 5% CO₂ and 95% air for two days.

(3) Labeled Ligands

Aqueous solutions of ligands labeled with commercially available [³H], [125I], [14C], [35S], etc. are stored at 4° C. or −20° C., and diluted to 1 μM with the assay buffer.

(4) Standard Ligand Solution

The ligand is dissolved in PBS containing 0.1% bovine serum albumin (Sigma Co.) to have a volume of 1 mM, which is stored at −20° C.

2. Assay Method

(1) CHO cells that have expressed the receptor protein of the present invention are cultured in a 12-well culture plate and washed twice with 1 ml of the assay buffer, and 490 μl of the assay buffer is added to each well.

(2) After adding 5 μl of a 10⁻³ to 10⁻¹⁰ M test compound solution, 5 μl of a labeled ligand is added thereto followed by reacting at room temperature for an hour. To examine the non-specific binding, 5 μl of a ligand is previously added in place of the test compound.

(3) The reaction solution is removed, and the wells are washed three times with 1 ml each of the wash buffer. The labeled ligand bound to the cells is dissolved in 0.2N NaOH-1% SDS, and mixed with 4 ml of liquid scintillator A (manufactured by Wako Pure Chemical Industries, Ltd.)

(4) The radioactivity is measured using a liquid scintillation counter (manufactured by Beckman Co.), and the percent maximum binding (PMB) is calculated according to the following equation.

PMB=[(B−NSB)/(B₀−NSB)]×100

PMB: Percent maximum binding

B: Value obtained in the presence of a test compound

NSB: Non-specific binding

B₀: Maximum binding

The compounds or salts thereof which are obtainable using the screening method or the screening kit of the present invention are compounds having the activity of altering the binding property between ligands and the receptor protein of the present invention. Specifically, such compounds include (a) compounds that have the G protein-coupled receptor-mediated cell-stimulating activities (e.g., the activities that promote or suppress arachidonic acid release, acetylcholine release, intracellular Ca²⁺, release, intracellular cAMP production, intracellular cGMP production, inositol phosphate production, alters in cell membrane potential, phosphorylation of intracellular proteins, activation of c-fos, pH reduction, etc.) (so-called agonists to the receptor protein of the present invention); (b) the compounds that do not have the cell-stimulating activities (so-called antagonists to the receptor protein of the present invention); (c) the compounds that increase the binding force between ligands and the receptor protein of the present invention; or (d) the compounds that decrease the binding force between ligands and the receptor protein of the present invention.

Examples of the compounds include peptides, proteins, non-peptide compounds, synthetic compounds, fermentation products, and the like. These compounds may be novel or known compounds.

The agonists to the receptor protein of the present invention have similar activities as the physiological activities possessed by the ligands to the receptor protein of the present invention. Therefore, these agonists are useful as safe and low-toxic pharmaceuticals depending on the ligand activities (for the prevention and/or treatment of, e.g., hypertension, autoimmune disease, heart failure, cataract, glaucoma, acute bacterial meningitis, acute myocardial infarction, acute pancreatitis, acute viral encephalitis, adult respiratory distress syndrome, alcoholic hepatitis, Alzheimer's disease, asthma, arteriosclerosis, atopic dermatitis, bacterial pneumonia, bladder cancer, fracture, breast cancer, bulimia, polyphagia, burn healing, uterine cervical cancer, chronic lymphocytic leukemia, chronic myelogenous leukemia, chronic pancreatitis, liver cirrhosis, cancer of the colon and rectum (colon cancer/rectal cancer), Crohn's disease, dementia, diabetic complications, diabetic nephropathy, diabetic neuropathy, diabetic retinopathy, gastritis, Helicobacter pylori bacterial infectious disease, hepatic insufficiency, hepatitis A, hepatitis B, hepatitis C, hepatitis, herpes simplex virus infectious disease, varicellazoster virus infectious disease, Hodgkin's disease, AIDS infectious disease, human papilloma virus infectious disease, hypercalcemia, hypercholesterolemia, hyperglyceridemia, hyperlipemia, infectious disease, influenza infectious disease, insulin dependent diabetes mellitus (type I), invasive staphylococcal infectious disease, malignant melanoma, cancer metastasis, multiple myeloma, allergic rhinitis, nephritis, non-Hodgkin's lymphoma, insulin-independent diabetes mellitus (type II), non-small cell lung cancer, organ transplantation, arthrosteitis, osteomalacia, osteopenia, osteoporosis, ovarian cancer, Behcet's disease of bone, peptic ulcer, peripheral vessel disease, prostatic cancer, reflux esophagitis, renal insufficiency, rheumatoid arthritis, schizophrenia, sepsis, septic shock, severe systemic fungal infectious disease, small cell lung cancer, spinal injury, stomach cancer, systemic lupus erythematosus, transient cerebral ischemia, tuberculosis, cardiac valve failure, vascular/multiple infarction dementia, wound healing, insomnia, arthritis, pituitary hormone secretion disorder, pollakiuria, uremia, neurodegenerative disease, etc.).

The agonists of the receptor protein of the present invention can suppress the physiological activities possessed by the ligands to the receptor protein of the present invention, and are thus useful as safe and low-toxic pharmaceuticals for suppressing the ligand activities.

The compounds that potentiate the binding force between the ligands and the receptor protein of the present invention are useful as safe and low-toxic pharmaceuticals for increasing the physiological activities possessed by the ligands to the receptor protein of the present invention (for the prevention and/or treatment of, e.g., hypertension, autoimmune disease, heart failure, cataract, glaucoma, acute bacterial meningitis, acute myocardial infarction, acute pancreatitis, acute viral encephalitis, adult respiratory distress syndrome, alcoholic hepatitis, Alzheimer's disease, asthma, arteriosclerosis, atopic dermatitis, bacterial pneumonia, bladder cancer, fracture, breast cancer, bulimia, polyphagia, burn healing, uterine cervical cancer, chronic lymphocytic leukemia, chronic myelogenous leukemia, chronic pancreatitis, liver cirrhosis, cancer of the colon and rectum (colon cancer/rectal cancer), Crohn's disease, dementia, diabetic complications, diabetic nephropathy, diabetic neuropathy, diabetic retinopathy, gastritis, Helicobacter pylori bacterial infectious disease, hepatic insufficiency, hepatitis A, hepatitis B, hepatitis C, hepatitis, herpes simplex virus infectious disease, varicellazoster virus infectious disease, Hodgkin's disease, AIDS infectious disease, human papilloma virus infectious disease, hypercalcemia, hypercholesterolemia, hyperglyceridemia, hyperlipemia, infectious disease, influenza infectious disease, insulin dependent diabetes mellitus (type I), invasive staphylococcal infectious disease, malignant melanoma, cancer metastasis, multiple myeloma, allergic rhinitis, nephritis, non-Hodgkin's lymphoma, insulin-independent diabetes mellitus (type II), non-small cell lung cancer, organ transplantation, arthrosteitis, osteomalacia, osteopenia, osteoporosis, ovarian cancer, Behcet's disease of bone, peptic ulcer, peripheral vessel disease, prostatic cancer, reflux esophagitis, renal insufficiency, rheumatoid arthritis, schizophrenia, sepsis, septic shock, severe systemic fungal infectious disease, small cell lung cancer, spinal injury, stomach cancer, systemic lupus erythematosus, transient cerebral ischemia, tuberculosis, cardiac valve failure, vascular/multiple infarction dementia, wound healing, insomnia, arthritis, pituitary hormone secretion disorder, pollakiuria, uremia, neurodegenerative disease, etc.).

The compounds that decrease the binding force between the ligands and the receptor protein of the present invention are useful as safe and low-toxic pharmaceuticals for reducing the physiological activities possessed by the ligands to the receptor protein of the present invention.

When the compound or its salts, which can be obtained by the screening method or screening kit of the present invention, are used as the pharmaceutical compositions described above, the compound or its salts are made into and used as pharmaceutical preparations following a conventional means. For example, the compound or its salts may be prepared into tablets, capsules, elixirs, microcapsules, sterile solutions, suspensions, etc., as in the pharmaceuticals containing the receptor protein of the present invention described above.

Since the pharmaceutical preparations thus obtained are safe and low toxic, they may be administered to human or mammals (e.g., rat, rabbit, sheep, swine, bovine, cat, dog, monkey, etc.).

The dose of the compound or its salts varies depending on subject to be administered, target organ, symptom, method for administration, etc.; for example, in oral administration, the dose is normally about 0.1 to about 100 mg, preferably about 1.0 to about 50 mg, more preferably about 1.0 to about 20 mg per day for the patient with hypertension (as 60 kg body weight). In parenteral administration, the single dose varies depending on subject to be administered, target organ, symptom, method for administration, etc., but it is advantageous to administer the compound or its salts intravenously to the patient with hypertension (as 60 kg body weight) at a daily dose of about 0.01 to about 30 mg, preferably about 0.1 to about 20 mg, more preferably about 0.1 to about 10 mg. For other animal species, the corresponding dose as converted per 60 kg weight can be administered.

(8) Prophylactic and/or Therapeutic Drugs for Various Diseases, Comprising the Compounds (Agonists, Antagonists) that Alter the Binding Property of the Receptor Protein of the Present Invention and Ligands

The compounds (agonists, antagonists) that alter the binding property of the receptor protein of the present invention and ligands can be used as prophylactic and/or therapeutic drugs for diseases associated with dysfunction of the receptor protein of the present invention (for the prevention and/or treatment of, e.g., hypertension, autoimmune disease, heart failure, cataract, glaucoma, acute bacterial meningitis, acute myocardial infarction, acute pancreatitis, acute viral encephalitis, adult respiratory distress syndrome, alcoholic hepatitis, Alzheimer's disease, asthma, arteriosclerosis, atopic dermatitis, bacterial pneumonia, bladder cancer, fracture, breast cancer, bulimia, polyphagia, burn healing, uterine cervical cancer, chronic lymphocytic leukemia, chronic myelogenous leukemia, chronic pancreatitis, liver cirrhosis, cancer of the colon and rectum (colon cancer/rectal cancer), Crohn's disease, dementia, diabetic complications, diabetic nephropathy, diabetic neuropathy, diabetic retinopathy, gastritis, Helicobacter pylori bacterial infectious disease, hepatic insufficiency, hepatitis A, hepatitis B, hepatitis C, hepatitis, herpes simplex virus infectious disease, varicellazoster virus infectious disease, Hodgkin's disease, AIDS infectious disease, human papilloma virus infectious disease, hypercalcemia, hypercholesterolemia, hyperglyceridemia, hyperlipemia, infectious disease, influenza infectious disease, insulin dependent diabetes mellitus (type I), invasive staphylococcal infectious disease, malignant melanoma, cancer metastasis, multiple myeloma, allergic rhinitis, nephritis, non-Hodgkin's lymphoma, insulin-independent diabetes mellitus (type II), non-small cell lung cancer, organ transplantation, arthrosteitis, osteomalacia, osteopenia, osteoporosis, ovarian cancer, Behcet's disease of bone, peptic ulcer, peripheral vessel disease, prostatic cancer, reflux esophagitis, renal insufficiency, rheumatoid arthritis, schizophrenia, sepsis, septic shock, severe systemic fungal infectious disease, small cell lung cancer, spinal injury, stomach cancer, systemic lupus erythematosus, transient cerebral ischemia, tuberculosis, cardiac valve failure, vascular/multiple infarction dementia, wound healing, insomnia, arthritis, pituitary hormone secretion disorder, pollakiuria, uremia, neurodegenerative disease, etc.).

When the compounds are used as prophylactic and/or therapeutic drugs for diseases associated with dysfunction of the receptor protein of the present invention, the compounds can be prepared into pharmaceutical preparations by a conventional means.

For example, the compounds can be administered orally in the form of a sugar-coated dragee, capsule, elixir, microcapsule, etc., or parenterally in the form of injectable preparations such as a sterile solution, a suspension, etc., in water, or together with other pharmaceutically acceptable liquid. For example, these preparations can be manufactured by mixing the compounds with physiologically acceptable known carrier, flavor, excipient, vehicle, antiseptic, stabilizer, binder, etc. in a unit dosage form required for generally approved drug preparations. The amount of the active ingredient in these preparations is set to an appropriate dose within the indicated range.

For the additives that may be mixed in tablets, capsules, etc., for example, binders such as gelatin, cornstarch, tragacanth or gum arabic, excipients such as crystalline cellulose, swelling agents such as corn starch, gelatin, alginic acid, etc., lubricants such as magnesium stearate, sweeteners such as sucrose, lactose, saccharin, etc., and flavors such as peppermint, akamono oil, cherry, etc. are used. When the dosage form is a capsule, a liquid carrier such as fat and oil may further be contained, in addition to the materials described above. Sterile compositions for injection can be formulated following the conventional preparation procedures such as dissolving or suspending the active substance in a vehicle, e.g., water for injection, naturally occurring vegetable oils, e.g., sesame oil, coconut oil, etc. As the aqueous solution for injection, for example, physiological saline, isotonic solutions (e.g., D-sorbitol, D-mannitol, sodium chloride, etc.) containing glucose and other aids are used. Appropriate dissolution-assisting agents, for example, an alcohol (e.g., ethanol), a polyalcohol (e.g., propylene glycol, polyethylene glycol), a nonionic surfactant (e.g., polysorbate 80™, HCO-50), etc. may be used in combination. For the oily solution, for example, sesame oil, soybean oil and the like are used, and dissolution-assisting agents such as benzyl benzoate, benzyl alcohol, etc. may be used in combination.

The prophylactic/therapeutic drugs described above may be formulated together with buffers (e.g., phosphate buffer, sodium acetate buffer), pain killers (e.g., benzalkonium chloride, procaine hydrochloride), stabilizers (e.g., human serum albumin, polyethylene glycol, etc.), preservatives (e.g., benzyl alcohol, phenol, etc.), antioxidants, and the like. The injection preparations prepared are usually filled in appropriate ampoules.

The pharmaceutical preparations thus obtained are safe and low-toxic, and can be administered to, for example, human and mammal (e.g., rats, rabbits, sheep, swine, bovine, cats, dogs, monkeys, etc.).

The dosage of the compounds or its salts differs depending on subject to be administered, target organ, symptom, administration method, etc. The dose of the compound or salts thereof varies depending on subject to be administered, target organ, symptom, method for administration, etc.; for example, in oral administration, the dose is generally about 0.1 to about 100 mg, preferably about 1.0 to about 50 mg, more preferably about 1.0 to about 20 mg per day for the patient with hypertension (as 60 kg body weight). In parenteral administration, the single dose varies depending on subject to be administered, target organ, symptom, method for administration, etc. but it is advantageous to administer the compound intravenously to the patient with hypertension (as 60 kg body weight) at a daily dose of about 0.01 to about 30 mg, preferably about 0.1 to about 20 mg, more preferably about 0.1 to about 10 mg. For other animal species, the corresponding dose as converted per 60 kg weight can be administered.

(9) Quantification of the Receptor Protein of the Present Invention, its Partial Peptide, or Salts Thereof

The antibody of the present invention is capable of specifically recognizing the receptor protein of the present invention and thus, can be used for quantification of the receptor protein of the present invention in a test sample fluid, in particular, for quantification by sandwich immunoassay. That is, the present invention provides, for example:

(i) a method for quantification of the receptor protein of the present invention in a test sample fluid, which comprises competitively reacting the antibody of the present invention, a test sample fluid and a labeled receptor protein, and measuring the ratio of the labeled receptor protein bound to said antibody; and,

(ii) a method for quantification of the receptor protein of the present invention in a test sample fluid, which comprises reacting a test sample fluid simultaneously or continuously with the antibody of the present invention immobilized on a carrier and a labeled antibody of the present invention, and then assaying the activity of the labeling agent on the insoluble carrier.

In the method (ii) described above, it is preferred that one antibody is capable of recognizing the N-terminal region of the receptor protein of the present invention, while another antibody is capable of recognizing the C-terminal region of the receptor protein of the present invention.

The monoclonal antibody to the receptor protein of the present invention (hereinafter sometimes referred to as the monoclonal antibody of the present invention) may be used to assay the receptor protein of the present invention. Moreover, the receptor protein can be detected by means of a tissue staining as well. For these purposes, the antibody molecule per se may be used or F(ab′)₂, Fab′ or Fab fractions of the antibody molecule may also be used. There is no particular limitation for the assay method using the antibody to the receptor protein of the present invention; any method may be used so far as it relates to a method, in which the amount of an antibody, antigen or antibody-antigen complex can be detected by a chemical or physical means, depending on the amount of an antigen (e.g., the amount of the receptor protein) in a test sample fluid to be assayed, and then calculated using a standard curve prepared by a standard solution containing the known amount of antigen. Advantageously used are, for example, nephrometry, competitive method, immunometric method and sandwich method; in terms of sensitivity and specificity, the sandwich method, which will be described later, is particularly preferred.

Examples of the labeling agent used in the assay method using the labeling substance are radioisotopes, enzymes, fluorescent substances, luminescent substances, etc. Examples of the radioisotope are [¹²⁵I], [¹³¹I], [³H], [¹⁴C], etc. Preferred examples of the enzyme are those that are stable and have a high specific activity, which include β-galactosidase, β-glucosidase, alkaline phosphatase, peroxidase and malate dehydrogenase. Examples of the fluorescent substance are fluorescamine, fluorescein isothiocyanate, etc. Examples of the luminescent substance are luminol, a luminol derivative, luciferin, lucigenin, etc. Furthermore, the biotin-avidin system may also be used for binding of an antibody or antigen to a labeling agent.

In the immobilization of antigens or antibodies, physical adsorption may be used. Alternatively, chemical binding that is conventionally used for immobilization of proteins or enzymes may be used as well. Examples of the carrier include insoluble polysaccharides such as agarose, dextran and cellulose; synthetic resins such as polystyrene, polyacrylamide and silicone; glass; etc.

In the sandwich method, a test sample fluid is reacted with an immobilized monoclonal antibody of the present invention (first reaction), then reacted with another labeled monoclonal antibody of the present invention (second reaction) and the activity of the labeling agent on the insoluble carrier is assayed, whereby the amount of the receptor protein of the present invention in the test sample fluid can be quantified. The first and second reactions may be carried out in a reversed order, simultaneously or sequentially with an interval. The type of the labeling agent and the method for immobilization may be the same as those described hereinabove.

In the immunoassay by the sandwich method, it is not always necessary that the antibody used for the labeled antibody and for the solid phase should be one type or one species but a mixture of two or more antibodies may also be used for the purpose of improving the measurement sensitivity, etc.

In the method for assaying the receptor protein of the present invention by the sandwich method according to the present invention, preferred monoclonal antibodies of the present invention used for the first and the second reactions are antibodies, which binding sites to the receptor protein are different from one another. Thus, the antibodies used in the first and the second reactions are those wherein, when the antibody used in the second reaction recognizes the C-terminal region of the receptor protein, the antibody recognizing the site other than the C-terminal regions, e.g., recognizing the N-terminal region, is preferably used in the first reaction.

The monoclonal antibody of the present invention may be used in an assay system other than the sandwich method, such as a competitive method, an immunometric method, a nephrometry, etc. In the competitive method, an antigen in a test sample fluid and a labeled antigen are competitively reacted with an antibody, then the unreacted labeled antigen (F) and the labeled antigen bound to the antibody (B) are separated (i.e., B/F separation) and the labeled amount of either B or F is measured to determine the amount of the antigen in the test sample fluid. In the reactions for such a method, there are a liquid phase method in which a soluble antibody is used as the antibody and the B/F separation is effected by polyethylene glycol while a second antibody to the antibody is used, and a solid phase method in which an immobilized antibody is used as the first antibody or a soluble antibody is used as the first antibody while an immobilized antibody is used as the second antibody.

In the immunometric method, an antigen in a test sample fluid and an immobilized antigen are competitively reacted with a given amount of a labeled antibody followed by separating the solid phase from the liquid phase; or an antigen in a test sample fluid and an excess amount of labeled antibody are reacted, then an immobilized antigen is added to bind an unreacted labeled antibody to the solid phase and the solid phase is separated from the liquid phase. Thereafter, the labeled amount of any of the phases is measured to determine the antigen amount in the test sample fluid.

In the nephrometry, the amount of insoluble sediment, which is produced as a result of the antigen-antibody reaction in a gel or in a solution, is measured. Even when the amount of an antigen in a test sample fluid is small and only a small amount of the sediment is obtained, a laser nephrometry utilizing laser scattering can be suitably used.

In applying each of those immunoassays to the assay method for the present invention, any special conditions or operations are not required to set forth. The assay system for the receptor protein or its salts of the present invention may be constructed in addition to conditions or operations conventionally used for each of the methods, taking into account the technical consideration of one skilled in the art. For the details of such conventional technical means, a variety of reviews, reference books, etc. may be referred to [for example, Hiroshi Irie (ed.): “Radioimmunoassay” (published by Kodansha, 1974); Hiroshi Irie (ed.): “Radioimmunoassay; Second Series” (published by Kodansha, 1979); Eiji Ishikawa, et al. (ed.): “Enzyme Immunoassay” (published by Igaku Shoin, 1978); Eiji Ishikawa, et al. (ed.): “Enzyme Immunoassay” (Second Edition) (published by Igaku Shoin, 1982); Eiji Ishikawa, et al. (ed.): “Enzyme Immunoassay” (Third Edition) (published by Igaku Shoin, 1987); “Methods in Enzymology” Vol. 70 (Immuochemical Techniques (Part A)); ibid., Vol. 73 (Immunochemical Techniques (Part B)); ibid., Vol. 74 (Immunochemical Techniques (Part C)); ibid., Vol. 84 (Immunochemical Techniques (Part D: Selected Immunoassays)); ibid., Vol. 92 (Immunochemical Techniques (Part E: Monoclonal Antibodies and General Immunoassay Methods)); ibid., Vol. 121 (Immunochemical Techniques (Part I: Hybridoma Technology and Monoclonal Antibodies))(all published by Academic Press); etc.]

As described above, the receptor protein or its salts of the present invention can be quantified with high sensitivity, using the antibody of the present invention.

Further by quantifying the receptor protein or its salts of the present invention in vivo using the antibody of the present invention, various diseases associated with dysfunction of the receptor protein of the present invention can be diagnosed.

Also, the antibody of the present invention can be employed for detecting the receptor protein of the present invention which may be present in a test sample fluid such as a body fluid, a tissue, etc. The antibody can also be used for the preparation of an antibody column for purification of the receptor protein of the present invention, detection of the receptor protein of the present invention in the fractions upon purification, analysis of the behavior of the receptor protein of the present invention in the cells under investigation, or the like.

(10) Method of Screening a Compound that Alters a Level of the Receptor Protein or its Partial Peptide of the Present Invention on the Cell Membrane

Since the antibody of the present invention can specifically recognize the receptor protein or its partial peptide or their salts of the present invention, it can be used in screening a compound that alters a level of the receptor protein or its partial peptide of the present invention on the cell membrane.

That is, the present invention provides, for example, the following methods.

(i) A method of screening a compound that alters the amount of the receptor protein or its partial peptide in cell membranes, which comprises separating the cell membrane fraction from (1) blood, (2) specific organs or (3) tissues or cells isolated from the organs of non-human mammals, which tissues or cells are destructed, and quantifying the receptor protein or its partial peptide of the present invention contained in the cell membrane fraction.

(ii) A method of screening a compound that alters the amount of the receptor protein or its partial peptide of the present invention in cell membranes, which comprises destructing a transformant or the like capable of expressing the receptor protein or its partial peptide of the present invention, then separating the cell membrane fraction, and quantifying the receptor protein or its partial peptide of the present invention contained in the cell membrane fraction.

(iii) A method of screening a compound that alters the amount of the receptor protein or its partial peptide in cell membranes, which comprises immunostaining (1) blood, (2) specific organs or (3) tissues or cells isolated from the organs of non-human mammals, which tissues or cells are sectioned, and then confirming the protein on the cell membrane by quantifying the staining intensity of the receptor protein on the cell surface.

(iv) A method of screening a compound that alters the amount of the receptor protein or its partial peptide in cell membranes, which comprises immunostaining a sectioned transformant or the like capable of expressing the receptor protein or its partial peptide of the present invention and confirming the protein on the cell membrane by quantifying the staining intensity of the receptor protein on the cell surface.

Specifically, the receptor protein or its partial peptide of the present invention contained in the cell membrane fraction are quantified as follows.

(i) Normal or non-human animals of disease models (e.g., mice, rats, rabbits, sheep, swine, bovine, cats, dogs, monkeys, etc., more specifically, dementia rats, obese mice, arteriosclerosis rabbits, tumor-bearing mice, etc.) receive administration of a drug (e.g., neurotropic drugs, hypotensive drugs, anticancer drugs, antiobestic drugs, etc.) or physical stress (e.g., soaking stress, electric shock, light and darkness, low temperatures, etc.), and the blood, specific organs (e.g., brain, liver, kidneys, etc.), or tissues or cells isolated from the organs are collected after lapse of a specified time. The obtained organs, tissues, cells, etc. are suspended in, for example, an appropriate buffer (e.g., Tris hydrochloride buffer, phosphate buffer, Hepes buffer, etc.) or the like, and the organs, tissues, or cells are destroyed, and the cell membrane fraction is obtained using a surfactant (e.g., Triton-X 100™, Tween 20™, etc.) or the like, and further using techniques including centrifugal separation, filtration, column fractionation, etc.

The cell membrane fraction refers to a fraction abundant in cell membranes obtained after destruction of the cells by publicly known methods. The cell destruction methods include cell squashing using a Potter-Elvehjem homogenizer, disruption using a Waring blender or Polytron (manufactured by Kinematica Inc.), disruption by ultrasonication, disruption by cell spraying through thin nozzles under an increased pressure using a French press, or the like. Fractionation of cell membranes is effected mainly by fractionation using a centrifugal force, such as centrifugation for fractionation and density gradient centrifugation. For example, cell disruption fluid is centrifuged at a low speed (500 rpm to 3,000 rpm) for a short period of time (normally about 1 to about 10 minutes), the resulting supernatant is then centrifuged at a higher speed (15,000 rpm to 30,000 rpm) normally for 30 minutes to 2 hours. The precipitate thus obtained is used as the membrane fraction. The membrane fraction is rich in the receptor protein expressed and membrane components such as cell-derived phospholipids, membrane proteins, etc.

The receptor protein or its partial peptides of the present invention contained in the cell membrane fraction can be quantified by, for example, the sandwich immunoassay and the Western blotting analysis using the antibodies of the present invention.

The sandwich immunoassay can be performed by the procedures described above, and the Western blotting can be performed by publicly known methods.

(ii) Transformants expressing the receptor protein or its partial peptide of the present invention are prepared in accordance with the method described above, and the receptor protein or its partial peptide of the present invention contained in the cell membrane fraction can be quantified.

Compounds that alter the amount of the receptor protein or its partial peptides of the present invention in cell membranes can be screened as follows.

(i) To normal or non-human mammals of disease models, a test compound is administered at a specified time before (30 minutes to 24 hours before, preferably 30 minutes to 12 hours before, more preferably 1 hour to 6 hours before), at a specified time after (30 minutes to 3 days after, preferably 1 hour to 2 days after, more preferably 1 hour to 24 hours after), or simultaneously with a drug, physical stress or the like. At a specified time (30 minute to 3 days, preferably 1 hour to 2 days, more preferably 1 hour to 24 hours) after administration of the test compound, the amount of the receptor protein or its partial peptide of the present invention in cell membranes are quantified thereby to effect screening.

(ii) Transformants are cultured in a conventional manner and a test compound is mixed in a culture medium. After a specified time (1 day to 7 days after, preferably 1 day to 3 days after, more preferably 2 days or 3 days after), the amount of the receptor protein or its partial peptide of the present invention contained in the cell membrane can be quantified thereby to effect screening.

Specifically, the receptor protein or its partial peptide of the present invention contained in the cell membrane fraction is confirmed as follows.

Specifically, the receptor protein or its partial peptide of the present invention contained in cell membrane fractions are confirmed as follows.

(iii) Normal or non-human mammals of disease models (e.g., mice, rats, rabbits, sheep, swine, bovine, cats, dogs, monkeys, more specifically, dementia rats, obese mice, arteriosclerotic rabbits, tumor-bearing mice, etc.) receive administration of a drug (e.g., neurotropic drugs, hypotensive drugs, anticancer drugs, antiobestic drugs) or physical stress (e.g., soaking stress, electric shock, light and darkness, low temperature, etc.) or the like, and the blood, specific organ (e.g., brain, liver, kidneys), or tissues or cells isolated from the organs are obtained after a certain period of time. Tissue sections are prepared from the obtained organs, tissues, cells, etc. in a conventional manner, followed by immunostaining using the antibody of the present invention. By quantifying the staining intensity of the receptor protein on the cell surface layer, the protein on the cell membrane can be confirmed thereby to confirm the amount of the receptor protein or its partial peptide of the present invention quantitatively or qualitatively.

(iv) The receptor protein or its partial peptide of the present invention can also be confirmed by the similar method using transformants expressing the receptor protein or its partial peptide of the present invention.

Compounds or salts thereof, which can be obtained by the screening method of the present invention, are compounds that alter the amount of the receptor protein or its partial peptide of the present invention, and are specifically; (a) compounds that increase the amount of the receptor protein or its partial peptide of the present invention in cell membranes thereby to potentiate the G protein-coupled receptor-mediated cell stimulating activities (e.g., the activities that promote or suppress arachidonic acid release, acetylcholine release, intracellular Ca²⁺ release, intracellular cAMP production, intracellular cGMP production, inositol phosphate production, changes in cell membrane potential, phosphorylation of intracellular proteins, activation of c-fos, pH reduction, and the like); and (b) compounds that reduce the cell stimulating activities by decreasing the amount of the receptor protein or its partial peptide of the present invention in cell membranes.

The compounds include peptides, proteins, non-peptide compounds, synthetic compounds, a fermentation products, etc. These compounds may be novel or publicly known compounds.

The compounds that potentiate the cell stimulating activities are useful as safe and low-toxic pharmaceuticals for increasing the physiological activities of the receptor protein of the present invention (for the prevention and/or treatment of, e.g., hypertension, autoimmune disease, heart failure, cataract, glaucoma, acute bacterial meningitis, acute myocardial infarction, acute pancreatitis, acute viral encephalitis, adult respiratory distress syndrome, alcoholic hepatitis, Alzheimer's disease, asthma, arteriosclerosis, atopic dermatitis, bacterial pneumonia, bladder cancer, fracture, breast cancer, bulimia, polyphagia, burn healing, uterine cervical cancer, chronic lymphocytic leukemia, chronic myelogenous leukemia, chronic pancreatitis, liver cirrhosis, cancer of the colon and rectum (colon cancer/rectal cancer), Crohn's disease, dementia, diabetic complications, diabetic nephropathy, diabetic neuropathy, diabetic retinopathy, gastritis, Helicobacter pylori bacterial infectious disease, hepatic insufficiency, hepatitis A, hepatitis B, hepatitis C, hepatitis, herpes simplex virus infectious disease, varicellazoster virus infectious disease, Hodgkin's disease, AIDS infectious disease, human papilloma virus infectious disease, hypercalcemia, hypercholesterolemia, hyperglyceridemia, hyperlipemia, infectious disease, influenza infectious disease, insulin dependent diabetes mellitus (type I), invasive staphylococcal infectious disease, malignant melanoma, cancer metastasis, multiple myeloma, allergic rhinitis, nephritis, non-Hodgkin's lymphoma, insulin-independent diabetes mellitus (type II), non-small cell lung cancer, organ transplantation, arthrosteitis, osteomalacia, osteopenia, osteoporosis, ovarian cancer, Behcet's disease of bone, peptic ulcer, peripheral vessel disease, prostatic cancer, reflux esophagitis, renal insufficiency, rheumatoid arthritis, schizophrenia, sepsis, septic shock, severe systemic fungal infectious disease, small cell lung cancer, spinal injury, stomach cancer, systemic lupus erythematosus, transient cerebral ischemia, tuberculosis, cardiac valve failure, vascular/multiple infarction dementia, wound healing, insomnia, arthritis, pituitary hormone secretion disorder, pollakiuria, uremia, neurodegenerative disease, etc.).

The compounds that decrease the cell stimulating activities are useful as safe and low-toxic pharmaceuticals for decreasing the physiological activities of the receptor protein of the present invention.

The compound or its salts, which can be obtained by the screening method of the present invention, are used as pharmaceutical compositions in accordance with conventional methods. For example, the compound or its salts may be prepared into tablets, capsules, elixirs, microcapsules, sterile solutions, suspensions, etc., as in the pharmaceuticals containing the receptor protein of the present invention described above.

Since the pharmaceutical preparations thus obtained are safe and low toxic, they may be administered to human or mammals (e.g., rat, rabbit, sheep, swine, bovine, cat, dog, monkey, etc.).

The dose of the compound or its salts varies depending on subject to be administered, target organ, symptom, method for administration, etc.; for example, in oral administration, the dose is normally about 0.1 to about 100 mg, preferably about 1.0 to about 50 mg, more preferably about 1.0 to about 20 mg per day for the patient with hypertension (as 60 kg body weight). In parenteral administration, the single dose varies depending on subject to be administered, target organ, symptom, method for administration, etc., but it is advantageous to administer the compound or its salts intravenously to the patient with hypertension (as 60 kg body weight) at a daily dose of about 0.01 to about 30 mg, preferably about 0.1 to about 20 mg, more preferably about 0.1 to about 10 mg. For other animal species, the corresponding dose as converted per 60 kg weight can be administered.

(11) Prophylactic and/or Therapeutic Drugs for Various Diseases Containing Compounds that Alter the Amount of the Receptor Protein or its Partial Peptide of the Present Invention in Cell Membranes

As described above, the receptor protein of the present invention is considered to play some important role in the body, such as the central function, etc. Therefore, compounds that alter the amount of the receptor protein or its partial peptide of the present invention in cell membranes can be used as prophylactic and/or therapeutic drugs for diseases associated with dysfunction of the receptor protein of the present invention.

When the compounds are used as prophylactic and/or therapeutic drugs for diseases associated with dysfunction of the receptor protein of the present invention, the compounds can be prepared into pharmaceutical preparations by a conventional means.

For example, the compounds can be administered orally in the form of a sugar-coated dragee, capsule, elixir, microcapsule, etc., or parenterally in the form of injectable preparations such as a sterile solution, a suspension, etc., in water, or together with other pharmaceutically acceptable liquid. For example, these preparations can be manufactured by mixing the compounds with physiologically acceptable known carrier, flavor, excipient, vehicle, antiseptic, stabilizer, binder, etc. in a unit dosage form required for generally approved drug preparations. The amount of the active ingredient in these preparations is set to an appropriate dose within the indicated range.

For the additives that may be mixed in tablets, capsules, etc., for example, binders such as gelatin, cornstarch, tragacanth or gum arabic, excipients such as crystalline cellulose, swelling agents such as corn starch, gelatin, alginic acid, etc., lubricants such as magnesium stearate, sweeteners such as sucrose, lactose, saccharin, etc., and flavors such as peppermint, akamono oil, cherry, etc. are used. When the dosage form is a capsule, a liquid carrier such as fat and oil may further be contained, in addition to the materials described above. Sterile compositions for injection can be formulated following the conventional preparation procedures such as dissolving or suspending the active substance in a vehicle, e.g., water for injection, naturally occurring vegetable oils, e.g., sesame oil, coconut oil, etc. As the aqueous solution for injection, for example, physiological saline, isotonic solutions (e.g., D-sorbitol, D-mannitol, sodium chloride, etc.) containing glucose and other aids are used. Appropriate dissolution-assisting agents, for example, an alcohol (e.g., ethanol), a polyalcohol (e.g., propylene glycol, polyethylene glycol), a nonionic surfactant (e.g., polysorbate 80™, HCO-50), etc. may be used in combination. For the oily solution, for example, sesame oil, soybean oil and the like are used, and dissolution-assisting agents such as benzyl benzoate, benzyl alcohol, etc. may be used in combination.

The prophylactic/therapeutic drugs described above may be formulated together with buffers (e.g., phosphate buffer, sodium acetate buffer), pain killers (e.g., benzalkonium chloride, procaine hydrochloride), stabilizers (e.g., human serum albumin, polyethylene glycol, etc.), preservatives (e.g., benzyl alcohol, phenol, etc.), antioxidants, and the like. The injection preparations prepared are usually filled in appropriate ampoules.

The pharmaceutical preparations thus obtained are safe and low-toxic, and can be administered to, for example, human and mammal (e.g., rats, rabbits, sheep, swine, bovine, cats, dogs, monkeys, etc.).

The dosage of the compounds or its salts differs depending on subject to be administered, target organ, symptom, administration method, etc. The dose of the compound or salts thereof varies depending on subject to be administered, target organ, symptom, method for administration, etc.; for example, in oral administration, the dose is generally about 0.1 to about 100 mg, preferably about 1.0 to about 50 mg, more preferably about 1.0 to about 20 mg per day for the patient with hypertension (as 60 kg body weight). In parenteral administration, the single dose varies depending on subject to be administered, target organ, symptom, method for administration, etc. but it is advantageous to administer the compound intravenously to the patient with hypertension (as 60 kg body weight) at a daily dose of about 0.01 to about 30 mg, preferably about 0.1 to about 20 mg, more preferably about 0.1 to about 10 mg. For other animal species, the corresponding dose as converted per 60 kg weight can be administered.

(12) Neutralization by Antibodies to the Receptor Protein, its Partial Peptide, or its Salts of the Present Invention

The neutralizing activity of antibodies to the receptor protein, its partial peptide, or its salts of the present invention refers to the activity of inactivating the signal transduction function in which the receptor protein takes part. Therefore, when the antibody has the neutralizing activity, the antibody can inactivate the signal transduction in which the receptor protein is involved, for example, inactivate the cell-stimulating activities mediated by the receptor protein (e.g., the activities that promote or suppress arachidonic acid release, acetylcholine release, intracellular Ca²⁺ release, intracellular cAMP production, intracellular cGMP production, inositol phosphate production, changes in cell membrane potential, phosphorylation of intracellular proteins, activation of c-fos, pH reduction, etc.). Thus, the antibody can be used for the prevention and/or treatment of diseases caused by overexpression of the receptor protein, etc.

(13) Preparation of Non-Human Animal Bearing the DNA Encoding the Receptor Protein of the Present Invention

Transgenic non-human mammals capable of expressing the receptor protein of the present invention can be prepared using the DNA of the present invention. Examples of non-human mammals include mammals (e.g., rats, mice, rabbits, sheep, swine, bovine, cats, dogs, monkeys, etc.) and the like (hereinafter merely referred to as animal).

To introduce the DNA of the present invention into a target animal, it is generally advantageous to employ the DNA as a gene construct ligated downstream a promoter capable of expressing the DNA in an animal cell. For example, when the mouse-derived DNA of the present invention is introduced, for example, the gene construct, in which the DNA is ligated downstream various promoters capable of expressing the DNA of the present invention derived from an animal that is highly homologous to the DNA of the present invention, is microinjected to mouse fertilized ova. Thus, the DNA-introduced animal capable of producing a high level of the receptor protein of the present invention can be produced. As the promoters, there may be used, e.g., a virus-derived promoter and a ubiquitous expression promoter such as metallothionein, etc. Preferably, promoters of NGF gene, enolase, etc. that are specifically expressed in the brain are used.

The transfer of the DNA of the present invention to the fertilized egg cell stage secures the presence of the DNA in all germ and somatic cells in the target animal. The presence of the receptor protein of the present invention in the germ cells in the DNA-introduced animal means that all germ and somatic cells contain the receptor protein of the present invention in all progenies of the animal. The progenies of the animal that took over the gene contain the receptor protein of the present invention in all germ and somatic cells.

The transgenic animal to which the DNA of the present invention has been transferred can be subjected to mating and breeding for generations under common breeding circumstance, as the DNA-carrying animal, after confirming that the gene can be stably retained. Moreover, male and female animals having the desired DNA are mated to give a homozygote having the transduced gene in both homologous chromosomes and then the male and female animals are mated so that such breeding for generations that progenies contain the DNA can be performed.

The transgenic animal to which the DNA of the present invention has been introduced is useful as the animal for screening of the agonists or antagonists to the receptor protein of the present invention, since the receptor protein of the present invention is abundantly expressed.

The transgenic animal to which the DNA of the present invention has been introduced can also be used as the cell sources for tissue culture. The receptor protein of the present invention can be analyzed by, for example, direct analysis of the DNA or RNA in the tissues from the DNA-transferred mouse of the present invention, or by analysis of the tissues containing the receptor protein of the present invention expressed from the gene. Cells from tissues containing the receptor protein of the present invention are cultured by the standard tissue culture technique and using these cells, the function of the cells from the tissues that are generally difficult to culture, for example, cells derived from the brain and peripheral tissues can be studied. Using these cells it is also possible to screen pharmaceuticals, for example, that enhance the function of various tissues. Where a highly expressing cell line is available, the receptor protein of the present invention can be isolated and purified from the cell line.

In the specification and drawings, when bases, amino acids, etc. are shown by abbreviations, the codes of bases and amino acids are denoted in accordance with the IUPAC-IUB Commission on Biochemical Nomenclature or by the codes conventionally used in the art, examples of which are shown below. With respect to amino acids that may have their optical isomers, L form is given, unless otherwise indicated.

| | |
|---|---|
| DNA | deoxyribonucleic acid |
| cDNA | complementary deoxyribonucleic acid |
| A | adenine |
| T | thymine |
| G | guanine |
| C | cytosine |
| RMA | ribonucleic acid |
| mRNA | messenger ribonucleic acid |
| dATP | deoxyadenosine triphosphate |
| dTTP | deoxythymidine triphosphate |
| dGTP | deoxyguanosine triphosphate |
| dCTP | deoxycytidine triphosphate |
| ATP | adenosine triphosphate |
| EDTA | ethylenediaminetetraacetic acid |
| SDS | sodium dodecyl sulfate |
| Gly | glycine |
| Ala | alanine |
| Val | valine |
| Leu | leucine |
| Ile | isoleucine |
| Ser | serine |
| Thr | threonine |
| Cys | cysteine |
| Met | methionine |
| Glu | glutamic acid |
| Asp | aspartic acid |
| Lys | lysine |
| Arg | arginine |
| His | histidine |
| Phe | phenylalanine |
| Tyr | tyrosine |
| Trp | tryptophan |
| Pro | proline |
| Asn | asparagine |
| Gln | glutamine |
| Me | methyl group |
| Et | ethyl group |
| Bu | butyl group |
| Ph | phenyl group |
| TC | thiazolidine-4(R)-carboxamide group |

Substituents, protecting groups and reagents generally used in this specification are presented as the codes below.

| | |
|---|---|
| Tos | p-toluenesulfonyl |
| CHO | formyl |
| Bzl | benzyl |
| Cl₂Bzl | 2,6-dichlorobenzyl |
| Bom | benzyloxyrnethyl |
| Z | benzyloxycarbonyl |
| Cl—Z | 2-chlorobenzyl oxycarbonyl |
| Br—Z | 2-bromobenzyl oxycarbonyl |
| Boc | t-butoxycarbonyl |
| DNP | dinitrophenol |
| Trt | trityl |
| Bum | t-butoxymethyl |
| Fmoc | N-9-fluorenyl methoxycarbonyl |
| HOBt | 1-hydroxybenztriazole |
| HOOBL | 3,4-dihydro-3-hydroxy-4-oxo-1,2,3- |
| | benzotriazine |
| HONB | 1-hydroxy-5-norbornene-2,3-dicarboxyimide |
| DCC | N,N′-dichlorohexylcarbodiimide |

The sequence identification numbers in the sequence listing of the specification indicate the following sequences.

[SEQ ID NO: 1]

This shows the amino acid sequence of the human leukocyte-derived novel receptor protein hTGR2L.

[SEQ ID NO: 2]

This shows the base sequence of cDNA encoding the human leukocyte-derived novel receptor protein hTGR2L having the amino acid sequence shown by SEQ ID NO: 1.

[SEQ ID NO: 3]

This shows the amino acid sequence of the human leukocyte-derived novel receptor protein hTGR2V.

[SEQ ID NO: 4]

This shows the base sequence of cDNA encoding the human leukocyte-derived novel receptor protein hTGR2V having the amino acid sequence shown by SEQ ID NO: 3.

[SEQ ID NO: 5]

This shows the base sequence of primer 1 used in EXAMPLE 1 later described.

[SEQ ID NO: 6]

This shows the base sequence of primer 2 used in EXAMPLE 1 later described.

[SEQ ID NO: 7]

This shows the base sequence of primer 3 used in EXAMPLE 3 later described.

[SEQ ID NO: 8]

This shows the base sequence of primer 4 used in EXAMPLE 3 later described.

[SEQ ID NO: 9]

This shows the base sequence of the probe used in EXAMPLE 3 later described.

Escherichia coli transformant TOP10F′/pCR2.1-hTGR2L bearing cDNA encoding human leukocyte-derived novel receptor protein hTGR2L shown by SEQ ID NO: 2, which was obtained in EXAMPLE 1 later described, has been deposited with the Ministry of International Trade and Industry, Agency of Industrial Science and Technology, National Institute of Bioscience and Human Technology (NIBH) under the Accession Number FERM BP-7013 since Feb. 2, 2000 and with Institute for Fermentation, Osaka (IFO) under the Accession Number IFO 16349 since Jan. 13, 2000.

Escherichia coli transformant TOP10F′/pCR2.1-hTGR2V bearing cDNA encoding human leukocyte-derived novel receptor protein hTGR2V shown by SEQ ID NO: 4, which was obtained in EXAMPLE 1 later described, has been deposited with the Ministry of International Trade and Industry, Agency of Industrial Science and Technology, National Institute of Bioscience and Human Technology (NIBH) under the Accession Number FERM BP-7014 since Feb. 2, 2000 and with Institute for Fermentation, Osaka (IFO) under the Accession Number IFO 16350 since Jan. 13, 2000.

Hereinafter, the present invention is described in detail with reference to EXAMPLES, but not intended to limit the scope of the present invention thereto. The gene manipulation procedures using Escherichia coli were performed according to the methods described in the Molecular Cloning.

### EXAMPLE 1

Cloning and Determination of the Base Sequence of cDNA Encoding the Human Leukocyte-Derived G Protein-Coupled Receptor Protein

Using human leukocyte cDNA (CLONTECH, Inc.) as a template and using two primers: primer 1 (SEQ ID NO: 5) and primer 2 (SEQ ID NO: 6), PCR reaction was performed. The composition of the reaction solution was 1/10 volume of the cDNA for the template, 1/50 volume of Advantage-HF Polymerase Mix (CLONTECH, Inc.), 0.5 μM each of primer 1 (SEQ ID NO: 5) and primer 2 (SEQ ID NO: 6) and 200 μM of dNTPs, and the buffer attached to the enzyme was added thereto to make the volume 25 μl. In the PCR reaction, the reaction solution was heated at 94° C. for 5 minutes; then a cycle set to heat at 94° C. for 30 seconds, 60° C. for 30 seconds and 68° C. for 2 minutes was repeated 35 times; and finally, elongation reaction was performed at 68° C. for 5 minutes. The PCR product was subcloned to plasmid vector pCR2.1 (Invitrogen, Inc.) in accordance with the instruction attached to the TA cloning kit (Invitrogen, Inc.), which was then transfected to Escherichia coli TOP10F′ and clones bearing the cDNA were selected on LB agar plates containing ampicillin. The base sequence of each clone was analyzed. As a result, the base sequences (SEQ ID NO: 2 and SEQ ID NO: 4) of the cDNA encoding the novel G protein-coupled receptor protein were obtained. These two sequences are different each other by one base at the 781^{st} residue. The amino acid sequences deduced from those are the base sequences represented by SEQ ID NOs: 1 and 3 wherein the 261^{st} residue of amino acid sequence is leucine or valine. The novel G protein coupled receptor proteins comprising these amino acid sequences are designated as hTGR2L and hTGR2V. Also, the two transformants were designated as Escherichia coli TOP10F′/pCR2.1-hTGR2L and Escherichia coli TOP10F′/pCR2.1-hTGR2V, respectively.

### EXAMPLE 2

Construction of hTGR2 Expression Vector

Using as templates pCR2.1-hTGR2L and pCR2.1-hTGR2V, PCR was carried out under the same conditions as in EXAMPLE 1, using SalI site-added primer 1 (5′-GTCGACATGCTGGCAGCTGCCTTTGCAGACTCTAAC-3′) and SpeI site-added primer 2 (5′-TACTAGTCTATTTAACACCTTCCCCTGTCTCTTGATC-3′). The PCR products obtained were digested with two restriction enzymes SalI and SpeI, and the digestion products were subcloned to pAKKO1.11H (Biochemica et Biophysica Acta, 1219 (1994) 251-259) similarly digested with two restriction enzymes SalI and SpeI. After transfection to Escherichia coli DH5α, clones bearing the cDNA were selected in LB agar medium containing ampicillin. The thus acquired transformants inserted with plasmids bearing cDNAs encoding hTGR2L and hTGR2V were named Escherichia coli DH5α/TGR2L and DH5α/TGR2V, respectively.

### EXAMPLE 3

Analysis on Expression Distribution of hTGR2 in Human Tissues

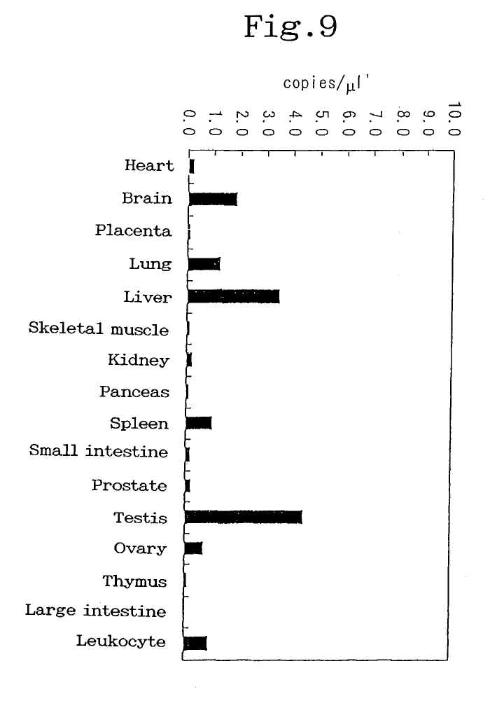

Expression distribution of hTGR2 in human tissues was analyzed using the TaqMan PCR method. Using Human Multiple Tissue cDNA panel (CLONTECH, Inc.) as a template, TaqMan PCR was carried out using as primers for PCR primer 3 (SEQ ID NO: 7 (5′-CTCCTGCTGTTTTCTGCACCT-3′)) and primer 4 (SEQ ID NO: 8 (5′-AGACAAACCAGCCTAGATCCCA-3′)) and probe (SEQ ID NO: 9 (5′-TCCGAGCTACGGCGTACTCCAAAAGTGT-3′)). The composition of the reaction solution in the reaction contained 12.5 μl of 2× Universal PCR Master Mix, 1 μl of 5 μM primer 1, 1 μl of 5 μM primer 2, 1 μl of 5 μM prove and 2 μl of the template, and 7.5 μl of distilled water to make the total volume 25 μl. PCR was performed at 50° C. for 2 minute and 95° C. for 10 minutes, and one cycle set to include 95° C. for 15 seconds and 60° C. for 1 minute was then repeated 40 times. Based on the results obtained, the results calculated as the number of copies per 1 ul of cDNA are shown in FIG. 9.

From the results it is seen that though the expression level of hTGR2 was not very high totally, hTGR2 was relatively highly expressed in testis, liver, brain, lung, spleen, leukocyte, ovary, etc.

Industrial Applicability

The receptor protein of the present invention, its partial peptide or salts thereof and polynucleotides (e.g., DNA, RNA and derivatives thereof) encoding the same can be used: (1) for determining ligands (agonists); (2) for acquiring antibodies and antisera; (3) for constructing the expression system of a recombinant receptor protein; (4) for developing a receptor-bound assay system and screening candidate compounds for drugs using the expression system above; (5) for designing drugs based on the comparison with ligand receptors having similar structures; (6) as reagents in preparing probes or PCR primers for gene therapy; (7) for constructing transgenic animals; or (8) as drugs such as gene preventives and remedies; etc.

What is claimed is:

1. A protein containing the same or substantially the same amino acid sequence as the amino acid sequence represented by SEQ ID NO: 1, or a salt thereof.

2. A partial peptide of the protein according to claim 1, or a salt thereof.

3. The protein or its salt according to claim 1, wherein the same or substantially the same amino acid sequence as the amino acid sequence represented by SEQ ID NO: 1 is the amino acid sequence represented by SEQ ID NO: 3.

4. A polynucleotide containing a polynucleotide encoding the protein according to claim 1.

5. The polynucleotide according to claim 4, which is a DNA.

6. The polynucleotide according to claim 4, bearing the base sequence represented by SEQ ID NO: 2 or SEQ ID NO: 4.

7. A recombinant vector containing the polynucleotide according to claim 4.

8. A transformant transformed by the recombinant vector according to claim 7.

9. A method for manufacturing the protein or its salt according to claim 1, which comprises culturing the transformant according to claim 8 and producing the protein according to claim 1.

10. An antibody to the protein according to claim 1 or the partial peptide according to claim 2, or a salt thereof.

11. The antibody according to claim 10, which is a neutralizing antibody to inactivate signal transduction of the protein according to claim 1.

12. A diagnostic composition comprising the antibody according to claim 10.

13. A ligand to the protein or its salt according to claim 1, which is obtainable using the protein according to claim 1 or the partial peptide according to claim 2, or a salt thereof.

14. A pharmaceutical composition comprising the ligand according to claim 13.

15. A method for determining a ligand to the protein or its salt according to claim 1, which comprises using the protein according to claim 1 or the partial peptide according to claim 2, or a salt thereof.

16. A method for screening a compound or a salt thereof that alters the binding property between a ligand and the protein or its salt according to claim 1, which comprises using the protein according to claim 1 or the partial peptide according to claim 2, or a salt thereof.

17. A kit for screening a compound or a salt thereof that alters the binding property between a ligand and the protein or its salt according to claim 1, comprising the protein according to claim 1 or the partial peptide according to claim 2, or a salt thereof.

18. A compound or a salt thereof that alters the binding property between a ligand and the protein or its salt according to claim 1, which is obtainable by the screening method according to claim 16 or using the screening kit according to claim 17.

19. A pharmaceutical composition comprising a compound or a salt thereof that alters the binding property between a ligand and the protein or its salt according to claim 1, which is obtainable using the screening method according to claim 16 or the screening kit according to claim 17.

20. A polynucleotide which is hybridizable to the polynucleotide according to claim 4 under high stringent conditions.

21. A polynucleotide containing a complementary base sequence to the polynucleotide according to claim 4, or a part thereof.

22. A method of quantifying mRNA of the protein according to claim 1, which comprises using the polynucleotide according to claim 4 or a part thereof.

23. A method of quantifying the protein of claim 1, which comprises using the antibody according to claim 10.

24. A method for diagnosis of diseases associated with the function of the protein according to claim 1, which comprises using the method of quantifying according to claim 22 or 23.

25. A method for screening a compound or a salt thereof that alters the expression level of the protein according to claim 1, which comprises using the method of quantifying according to claim 22.

26. A method for screening a compound or a salt thereof that alters the amount of the protein according to claim 1 in a cell membrane, which comprises using the method of quantifying according to claim 23.

27. A compound or a salt thereof that alters the expression level of the protein according to claim 1, which is obtainable using the method of quantifying according to claim 25.

28. A compound or a salt thereof that alters the amount of the protein according to claim 1 in a cell membrane, which is obtainable using the method of quantifying according to claim 26.
